(19) United States
(12) Patent Application Publication
(10) Pub. No.: US 2008/0269102 A1
(43) Pub. Date: Oct. 30, 2008

(54) 3-(2-Alkoxycarbonyloxy-Phenyl) Acrylic Acid Esters And Their Use As Precursors For The Delivery Of Olfactory Compounds

(75) Inventors: Felix Flachsmann, Duebendorf (CH); Jean-Pierre Bachmann, Wadenswil (CH)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 10/597,404

(22) PCT Filed: Feb. 10, 2005
(86) PCT No.: PCT/CH2005/000074

§ 371 Date: Jul. 24, 2006

(30) Foreign Application Priority Data
Feb. 12, 2004 (GB) 0403115.9

(51) Int. Cl. A61K 8/37; C07C 69/76; A61Q 13/00; C12P 7/62

(52) U.S. Cl. 512/21; 560/81; 435/135

(57) ABSTRACT

A compound of formula (I), their use as precursors and a method of their production

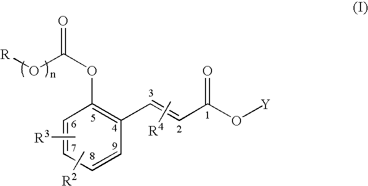

wherein n, Y, R, R², R³, and R⁴ has the same meaning as given in the specification.

The present invention refers to 3-(2-alkoxycarbonyloxy-phenyl)acrylic acid esters and their use as precursors for the delivery of olfactory compounds. This invention relates furthermore to a method of their production and to consumer products comprising them.

A principal strategy currently employed in imparting odors to consumer products is the admixing of the fragrance directly into the product. There are, however, several drawbacks to this strategy. The fragrance material can be too volatile and/or too soluble in water, resulting in fragrance loss during manufacturing, storage, and use. Many fragrance materials are also unstable over time. This again results in loss during storage. In many consumer products it is desirable for the fragrance to be released slowly over time. Microencapsulation and inclusion complexes with cyclodextrins have been used to help decrease volatility, improve stability and provide slow-release properties. However, these methods are for a number of reasons often not successful. In addition, cyclodextrins can be too expensive for use in many applications. It is therefore desirable to have a fragrance delivery system which is capable of releasing the fragrant compound or compounds in a controlled manner, maintaining a desired odour or fragrance over a prolonged period of time.

The principle of using precursors for the delivery of fragrance compounds appeared for the first time some years ago in the literature. The use of 3-(2-hydroxyaryl)acrylic acid esters (compound A below) is described in EP 0 936 211. This delivery system releases one or more olfactory compounds upon exposure to light. Using this system in various consumer products leads to a prolonged release of the fragrant compound(s). Unfortunately, the use of 3-(2-hydroxyaryl)acrylic acid esters may lead to discoloration, such as yellow discoloration, not only of consumer products, such as laundry care products, e.g. fabric softeners and detergents, comprising it, but it also may lead to discoloration of the substrate, for example, the fabric to which the product is applied during the washing cycle or rinse cycle. Discoloration of a product such as a fabric in general is not desired, thus there still remains a need for precursors having the advantageous ability to release one or more olfactory compounds, but without causing discoloration.

It has now been found that certain 3-(2-alkoxycarbonyloxy-phenyl)acrylic acid esters have the ability to release one or more olfactory compounds without showing discoloration to be visible to the naked eye. Surprisingly, it has been found that certain phenol protecting groups, in particular esters and carbonates, have the ability of rendering the prior art compounds (A)

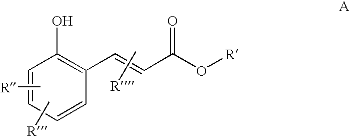

color-stable in consumer products comprising them.

Thus, it is believed, without restricting the invention in any way, that the free phenolic hydroxyl group of the prior art compounds, as described for example in EP 0 936 211 and WO 03/022978, is responsible for the discoloration.

Accordingly, a first aspect of the present invention refers to the use of a compound of formula (I) as precursor for olfactory compounds

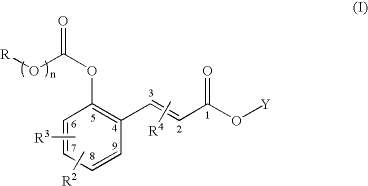

wherein the acrylic acid ester double bound is of the E configuration;

n is zero or 1;

Y is —CR⁵R⁶R⁷, wherein R⁵, R⁶ and R⁷ are independently hydrogen or a C₁-C₁₈, preferably C₁-C₁₀ hydrocarbon residue of which preferably at least one residue R⁵, R⁶ and R⁷ is not hydrogen, and the sum of all carbon atoms (R⁵+R⁶+R⁷) is not greater than 18, preferably the sum of all carbon atoms is between 6 and 15; or

Y is —CR⁵R⁶R⁷, wherein R⁵, R⁶ and R⁷ are independently hydrogen or a C₁-C₁₈ preferably C₁-C₁₀, hydrocarbon residue containing one or more atoms/groups selected from O, N and C(O), of which preferably at least one residue R⁵, R⁶ and R⁷ is not hydrogen, and the sum of all carbon atoms (R⁵+R⁶+R⁷) is not greater than 18, preferable the sum of all carbon atoms is between 6 and 15, for example Y is 2-(2-butoxy-ethoxy)-ethyl; or

Y is —CR⁸═CR⁹R¹⁰, wherein R⁸, R⁹ and R¹⁰ are independently hydrogen or a C₁-C₁₈, preferably C₁-C₁₀, hydrocarbon residue, of which preferably at least one of the residues R⁸, R⁹ and R¹⁰ is not hydrogen, the geometry of the enol double bond is E or Z, and the sum of all carbon atoms (R⁸+R⁹+R¹⁰) is not greater than 18, preferable the sum of all carbon atoms is between 6 and 15; or

Y is —CR⁸═CR⁹R¹⁰, wherein R⁸, R⁹ and R¹⁰ are independently hydrogen or a C₁-C₁₈, preferably C₁-C₁₀, hydrocarbon residue containing one or more atoms/groups selected from O, N and C(O), of which preferably at least one of the residues R⁸, R⁹ and R¹⁰ is not hydrogen, the geometry of the enol double bond is E or Z, and the sum of all carbon atoms (R⁸+R⁹+R¹⁰) is not greater than 18, preferable the sum of all carbon atoms is between 6 and 15;

R² and R³ are independently hydrogen; C₁-C₆ alkyl, e.g methyl, ethyl, iso-propyl, n-butyl, tert-butyl; C₁-C₆ alkoxy residue, e.g. methoxy, ethoxy; —NO₂; —NH₂; —NHCO₂CH₃; —N(C₁-C₆ alkyl)₂, e.g. dimethylamino, diethylamino; —N(hydroxyalkyl)₂, e.g. di(hydroxyethyl)amino, di(hydroxypropyl)amino; —NHC(O)—(C₁-C₈ alkyl) or —NHC(O)—(C₃-C₈ aryl), e.g. —NHC(O)-methyl or —NHC(O)-phenyl; or

R² and R³ are attached at the positions C(6,7), C(7,8), or C(8,9), and form together with the carbon atoms to which they are attached a dioxolane ring or a dioxane ring;

R⁴ in 2- or 3-position is hydrogen; C₁-C₄ alkyl, e.g. methyl, ethyl, tert-butyl; C₂-C₄ alkenyl, e.g. vinyl, propenyl; C₃-C₆ cycloalkyl, e.g. cyclopropyl, cyclopentyl, cyclohexyl; or —CN; and

if n is zero, R is a C₁-C₂₄, preferably C₁-C₁₈, hydrocarbon residue, e.g. methyl, ethyl or phenyl; or C₁-C₂₄, preferably C₁-C₁₈, hydrocarbon residue containing one or more heteroatoms selected from N, O and Si; or

if n is 1, R is a C₁-C₂₅, preferably C₁-C₁₈, hydrocarbon residue; a C₁-C₂₅ hydrocarbon residue containing one or more atoms/groups selected from N, O, Si, and C(O); or C₁-C₂₅, preferably C₁-C₁₈, hydrocarbon residue substituted by an ionic substituent of the formula N(R²⁰)₃⁺, in which R²⁰ is the residue of an alkyl group with 1 to 18 carbon atoms, preferably 1 to 8 carbon atoms, such as trimethylammonium, or tributylammonium; or R is a monovalent residue of the formula (I)

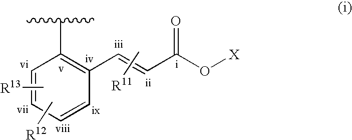

wherein
X is —CR¹⁴R¹⁵R¹⁶, wherein R¹⁴, R¹⁵ and R¹⁶ are independently hydrogen or a C₁-C₁₈, preferably C₁-C₁₀, hydrocarbon residue, of which preferably at least one residue R¹⁴, R¹⁵ and R¹⁶ is not hydrogen, and the sum of all carbon atoms (R¹⁴+R¹⁵+R¹⁶) is not greater than 18, preferable the sum of all carbon atoms is between 6 and 15; or
X is —CR¹⁴R¹⁵R¹⁶, wherein R¹⁴, R¹⁵ and R¹⁶ are independently hydrogen or a C₁-C₁₈, preferably C₁-C₁₀, hydrocarbon residue containing one or more atoms/groups selected from O, N and C(O), of which preferably at least one residue R¹⁴, R¹⁵ and R¹⁶ is not hydrogen, and the sum of all carbon atoms (R¹⁴+R¹⁵+R¹⁶) is not greater than 18, preferable the sum of all carbon atoms is between 6 and 15, for example X is 2-(2-butoxy-ethoxy)-ethyl; or
X is CR¹⁷=CR¹⁸R¹⁹, wherein R¹⁷, R¹⁸ and R¹⁹ are independently hydrogen or a C₁-C₁₈, preferably C₁-C₁₀, hydrocarbon residue, of which preferably at least one of the residues R¹⁷, R¹⁸ and R¹⁹ is not hydrogen, the geometry of the enol double bond is E or Z, and the sum of all carbon atoms (R¹⁷+R¹⁸+R¹⁹) is not greater than 18, preferable the sum of all carbon atoms is between 6 and 15; or
X is —CR¹⁷═CR¹⁸R¹⁹, wherein R¹⁷, R¹⁸ and R¹⁹ are independently hydrogen or a C₁-C₁₈, preferably C₁-C₁₀, hydrocarbon residue containing one or more atoms/groups selected from O, N and C(O), of which preferably at least one of the residues R¹⁷, R¹⁸ and R¹⁹ is not hydrogen, the geometry of the enol double bond is E or Z, and the sum of all carbon atoms (R¹⁷+R¹⁸+R¹⁹) is not greater than 18, preferable the sum of all carbon atoms is between 6 and 15;
R¹² and R¹³ are independently hydrogen; C₁-C₆ alkyl, e.g methyl, ethyl, iso-propyl, n-butyl, tert-butyl; C₁-C₆ alkoxy residue, e.g. methoxy, ethoxy; —NO₂; —NH₂; —NHCO₂CH₃; —N(C₁-C₆ alkyl)₂, e.g. dimethylamino, diethylamino; N(hydroxyalkyl)₂, e.g. di(hydroxyethyl)amino, di(hydroxypropyl)amino; —NHC(O)—(C₁-C₈ alkyl); or —NHC(O)—(C₃-C₈ aryl), e.g. —NHC(O)-methyl or —NHC(O)-phenyl; or
R¹² and R¹³ are attached at the positions C(vi,vii), C(vii,viii), or C(viii,ix), and form together with the carbon atoms to which they are attached a dioxolane ring or a dioxane ring;
R¹¹ in ii- or iii-position is hydrogen; C₁-C₄ alkyl, e.g. methyl, ethyl, tert-butyl; C₂-C₄ alkenyl, e.g. vinyl, propenyl; C₃-C₆ cycloalkyl, e.g. cyclopropyl, cyclopentyl, cyclohexyl; or —CN.

As used in relation to the compounds of formula (I) “hydrocarbon residue” unless otherwise indicated refers to alkyl, alkenyl, alkynyl, cycloalkyl, cycloalkenyl alkylcycloalkyl, alkenylcycloalkyl, alkenylcycloalkenyl, aryl, alkylaryl or arylalkyl, and “hydrocarbon residues containing one or more atoms/groups selected from O, N and C(O),” refers to alkyl, alkenyl, alkynyl, cycloalkyl, cycloalkenyl, alkylcycloalkyl, alkenylcycloalkyl, alkenylcycloalkenyl, aryl, alkylaryl or arylalkyl wherein one or more carbon atoms are replaced by O, N and/or C(O).

As used herein by “olfactory compound” is meant a molecule having an odour, preferably a pleasant odour, detectable by a human. As used herein, the terms “olfactory” and “fragrant” are used interchangeably, and refer to the same compounds.

Compounds of formula (I) are preferred wherein n is 1, and Y is the residue of a fragrant alcohol HO—CR⁵R⁶R⁷ or the residue of the enol form of a fragrant aldehyde of the formula O═(CH)—CHR⁹R¹⁰, or the residue of the enol form of a fragrant ketone of the formula O═(CR⁸)—CHR⁹R¹⁰ and if R is the monovalent residue of formula (I), X is the residue of a fragrant alcohol HO—CR¹⁴R¹⁵R¹⁶ or the residue of the enol form of a fragrant aldehyde of the formula O═(CH)—CHR¹⁸R¹⁹ or the residue of the enol form of a fragrant ketone of the formula O═(CR¹⁷)—CHR¹⁸R¹⁹.

Even more preferred are compounds of the present invention wherein n is 1, Y is the residue of a fragrant alcohol HO—CR⁵R⁶R⁷ or the residue of the enol form of a fragrant aldehyde of the formula O═(CH)—CHR⁹R¹⁰ or the residue of the enol form of a fragrant ketone of the formula O═(CR⁸)—CHR⁹R¹⁰, and R is selected from methyl, ethyl, propyl, butyl, pentyl, 2-ethylhexyl, cyclopentyl, cyclohexyl or the residue of a fragrant alcohol.

For the purpose of the present invention the term “fragrant alcohol” is defined herein as any alcohol having a molecular weight between 46 and 400, preferably between 100 and 300.

Examples of fragrant alcohols of the formula HO—CR⁵R⁶R⁷ and HO—CR¹⁴R¹⁵R¹⁶ include: amyl alcohol; hexyl alcohol*; 2-hexyl alcohol*; heptyl alcohol*; octyl alcohol*; nonyl alcohol*; decyl alcohol*; undecyl alcohol*; lauryl alcohol*; myristic alcohol; 3-methyl-but-2-en-1-ol*; 3-methyl-1-pentanol; cis-3-hexenol*; cis-4-hexenol*; 3,5,5-trimethyl-hexanol; 3,4,5,6,6-pentamethylheptan-2-ol*; citronellol*; geraniol*; oct-1-en-3-ol; 2,5,7-trimethyl-octan-3-ol; 2-cis-3,7-dimethyl-2,6-octadien-1-ol; 6-ethyl-3-methyl-5-octen-1-ol*; 3,7-dimethyl-oct-3,6-dienol*; 3,7-dimethyloctanol*; 7-methoxy-3,7-dimethyl-octan-2-ol*; cis-6-nonenol*; 5-ethyl-2-nonanol; 6,8-dimethyl-2-nonanol*; 2,2,8-trimethyl-7(8)-nonene-3-ol; nona-2,6-dien-1-ol; 4-methyl-3-decen-5-ol*; dec-9-en-1-ol **; benzylalcohol; 2-methyl-undecanol; 10-undecen-1-ol; 1-phenyl-ethanol*; 2-phenyl-ethanol*; 2-methyl-3-phenyl-3-propenol; 2-phenyl-propanol*; 3-phenyl-propanol*; 4-phenyl-2-butanol; 2-methyl-5-phenyl-pentanol*; 2-methyl-4-phenyl-pentanoi*; 3-methyl-5-phenyl-pentanol*; 2-(2-methylphenyl)ethanol*; 4-(1-methylethyl)benzene-methanol; 4-(4-hydroxyphenyl)-butan-2-one*; 2-phenoxy-ethanol*; 4-(1-methylethyl)-2-hydroxy-1-methyl benzene; 2-methoxy-4-methyl-phenol; 4-methyl-phenol; anisic alcohol*; p-tolyl alcohol*; cinnamic alcohol*; vanillin*; ethyl vanillin*; eugenol*; isoeugenol*; thymol; anethol*; decahydro-2-naphthalenol; borneol*; cedrenol*; farnesol*; fenchyl alcohol*; menthol*; 3,7,11-trimethyl-2,6,10-dodecatrien-1-ol; alpha ionol*; tetrahydro ionol*; 2-(11-dimethylethyl)cyclohexanol*; 3-(1,1-dimethylethyl)cyclohexanol*; 4-(1,1-dimethylethyl)cyclohexanol*; 4-isopropyl-cyclohexanol; 6,6-dimethyl-bicyclo[3.3.1]hept-2-ene-2-ethanol; 6,6-dimethyl-bicyclo[3.1.1]hept-2-ene-methanol*; p-menth-8-en-3-ol*; 3,3,5-trimethyl-cyclohexanol; 2,4,6-trimethyl-3-cyclohexenyl-methanol*; 4-(1-methylethyl)cyclohexyl-methanol*; 4-(1,1-dimethylethyl)cyclohexanol; 2-(1,1-dimethylethyl)cyclohexanol; 2,2,6-trimethyl-alpha-propyl-cyclohexane propanol*; 5-(2, 2,3-trimethyl-3-cyclopentenyl)-3-methylpentan-2-ol*; 3-methyl-5-(2,2,3-trimethylcyclopentyl-3-enyl)pent-4-en-2-ol*; 2-ethyl-4-(2,2,3-trimethylcyclopentyl-3-enyl)but-2-en-1-ol*; 4-(5,5,6-trimethylbicyclo[2.2.1]hept-2-yl)cyclohexanol*; 2-(2-methylpropyl)-4-hydroxy-4-methyl-tetrahydropyran*; 2-cyclohexyl-propanol*; 2-(1,1-dimethylethyl)-4-methyl-cyclohexanol*; 1-(2-tert-butyl-cyclohexyloxy)-2-butanol*; 1-(4-isopropyl-cyclohexyl)ethanol*; 2,6-dimethyl-oct-7-en-2-ol**; 2,6-dimethyl-heptan-2-ol**; and 3,7-dimethyl-octa-1,6-dien-3-ol**;

whereby * indicates the preferred alcohols and ** indicate the more preferred alcohols.

For the purpose of the present invention the term “fragrant aldehyde” is defined herein as any aldehyde having a molecular weight between 100 and 450, preferably between 120 and 300.

Examples of fragrant aldehydes of the formula O═(CH)—CHR⁹R¹⁰ and O═(CH)—CHR¹⁸R¹⁹ include:

2,6,10-trimethylundec-9-enal*; 1,2,3,4,5,6,7,8,-octahydro-8,8-dimethyl-2-napthalenecar-boxaldehyde; tridecanal; 2-[4-(1-methylethyl)phenyl]ethanal; 2,4-dimethyl-cyclohex-3-ene-1-carboxaldehyde*; 4-carboxaldehyde-1,3,5-trimethyl-cyclohex-1-ene*; 1-carboxaldehyde-2,4-dimethyl-cyclohex-3-ene*; 1-carboxaldehyde-4-(4-hydroxy-4-methylpentyl)cyclohex-3-ene*; 3,5,5-trimethyl-hexanal; heptanal*; 2,6-dimethyl-hept-5-enal*; decanal**; dec-9-enal; dec-4-enal; 2-methyldecanal*; undec-10-enal**; undecanal*; dodecanal**; 2-methyl-undecanal**; tridecanal; octanal**; nonanal*; 3,5,5-trimethylhexanal; undec-9-enal**; 2-phenyl-propanal*; 4-methyl-phenyl-acetaldehyde*; 3,7-dimethyl-octanal*; dihydroxy-3,7-dimethyl-octanal*; 2,6-dimethyl-oct-5-enal; 2-[4-(1-methylethyl)phenyl]ethanal*; 3-(3-isopropyl-phenylbutanal**; 2-(3,7-dimethyoct-6-enoxy)ethanal; 1-carboxaldehyde-4-(4-methyl-3-pentenyl)cyclohex-3-ene*; 2,3,5,5,-tetramethyl-hexanal; longifolic aldehyde; 2-methyl-4-(2,6,6-trimethylcyclohex-2-en-1-yl)butanal*; 2-methyl-3-(4-tert-butylphenyl)propanal** 4-(1,1-dimethyl-ethyl)benzene-propanal*; 2-[4-(1-methyl-ethyl)-phenyl]propanal; alpha-methyl-1,3-benzodioxole-5-propanal*; 3,7-dimethyl-oct-6-enal*; 2-methyl-3-(4-isopropylphenyl)-propionaldehyde*; 4-(4-hydroxy-4-methyl-pentyl)cyclohex-3-en-1-carboxaldehyde**; alpha-methyl-1,3-benzodioxole-5-propanal*; 1-carboxaldehyde-4-(1,1-dimethylethyl)-cyclohexane; 4-(octahydro-4,7-methano-5H-inden-5-ylidene)butanal; and [(3,7-dimethyl-6-octenyl)-oxy]acetaldehyde**;

whereby * indicates the preferred aldehydes and ** indicate the more preferred aldehydes.

For the purpose of the present invention the term “fragrant ketone” is defined herein as any ketone having a molecular weight between 100 and 450, preferably between 120 and 350.

Examples of fragrant ketones of the formula O═(CR⁸)—CHR⁹R¹⁰ and O═(CR¹⁷)—CHR¹⁸R¹⁹ include:

2-heptyl-cyclopentanone; 2,2,6,10-tetramethyltricyclo-[5.4.0.0(6, 10)]undecan-4-one benzylacetone*; carvone*; 1,2,3,5,6,7-hexahydro-1,1,2,3,3,-pentamentyl-4H-inden-4-one*; methyl heptenone*; dimethyl octenone*; 2-(butan-2-yl)cyclohexanone*; 2-hexyl-cyclopent-2-en-1-one*; 2-(1-methylethyl)-5-methyl-cyclohexanone*; 2-(2-methylethyl)-5-methyl-cyclohexanone*; 3-methyl-cyclopentadecanone; 4-tert-pentyl-cyclohexanone*; 3-oxo-2-pentyl-cyclopentane-acetic acid methyl ester**; 1-(1,2,3,4,5,6,7,8-octahydro-2,3,8,8-tetramethyl-2-naphthalenyl)ethanone*; and 3-methyl-5-propyl-cyclohex-2-en-1-one*;

whereby * indicates the preferred ketones and ** indicate the more preferred ketone.

Another embodiment relates to compounds of formula (I) wherein n is 1 and R and Y have the same meaning as given above and
I) R², R³ and R⁴ are H;
II) R² and R³ is H, and R⁴ is methyl or —CN at C(2) or C(3), or phenyl at C(3);
III) R² is H, R³ is methyl, ethyl, propyl, or isopropyl at either C(6) to C(8) or methoxy, ethoxy, propyloxy at either C(6) to C(8), and R⁴ is H, methyl or —CN at C(2) or C(3), or phenyl at C(3);
IV) R² and R³ is methyl at positions C(6,7), C(6,8), C(6,9), C(7,8), or C(8,9); or R² and R³ is methoxy at C(7,9), and R⁴ is H, methyl or —CN at C(2) or C(3), or phenyl at C(3);
V) R⁴ is H, methyl or —CN at C(2) or C(3), or phenyl at C(3), and R² is methyl at C(6) and R³ is isopropyl at C(9), or R² is isopropyl at C(6) and R³ is methyl at C(9).

Compounds of formula (I) are preferred wherein n is 1, R and Y have the same meaning as given above, R⁴ is hydrogen or methyl at position C(2) or C(3), and R² and R³ is hydrogen, or R² is hydrogen and R³ is 7-methoxy, or R² is hydrogen and R³ is 6-methyl, or R² is hydrogen and R³ is 7-methyl, or R² is hydrogen and R³ is 8-methyl, or R² is hydrogen and R³ is 6-tert-butyl, or R² is 6-tert-butyl and R³ is 8-tert-butyl.

The release of the active substances occurs in two successive steps via hydrolysis, preferably in the presence of enzymes, followed by photoisomerisation/lactonization, as shown in Scheme 1. Due to the two different consecutive cleavage mechanisms, it is possible to control the release of an olfactory compound at two different stages of the drying process of a substrate to which compounds of the present invention are applied. That is, for example, the release of an alcohol of the formula ROH (IV) after spin-drying of a fabric in a first step and the release of an alcohol (III), or for Y≡CR⁸═CR⁹R¹⁰ a ketone or aldehyde by tautomerisation, and a coumarin of formula (IIa), when exposed to UV light, e.g. sunlight during line-drying. In other words, a first boost of fragrance is perceivable when the washing machine is opened and a second boost of fragrance is perceivable during the line-drying process, if exposed to UV-light.

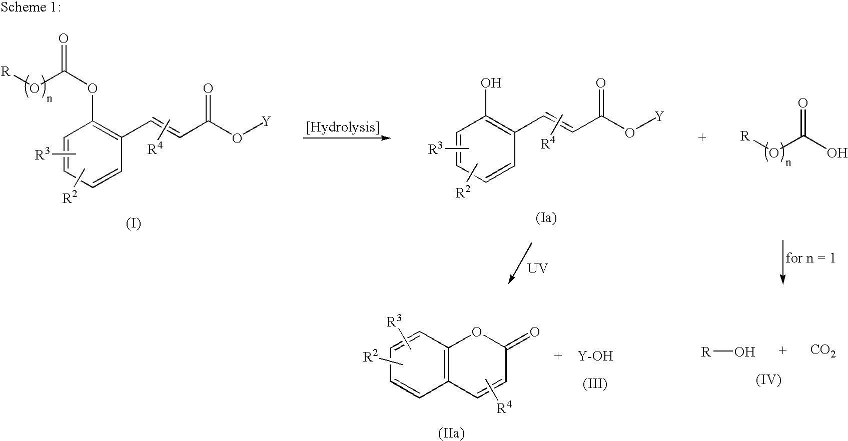

Accordingly, another aspect of the present invention is a process of providing an olfactory compound to a substrate comprising the steps:
a) cleaving a compound of formula (I) by hydrolysis resulting in a compound of formula (Ia); followed by
b) cleaving the compound of formula (Ia) of step a under activating conditions in the presence of light resulting in a coumarin (IIa)

In a preferred embodiment, at least the coumarin (IIa) and one of the alcohols (III, IV) are olfactory compounds. Even more preferred are compounds according to the present invention capable of releasing two coumarins (IIa, IIb), herein referred to as Type-II compounds, i.e. compounds of formula (I) wherein n is 1 and R is a monovalent residue of the formula (I) as shown in Scheme 2. Thus Type-II compounds of formula (I) can yield under activating conditions up to four different olfactory compounds.

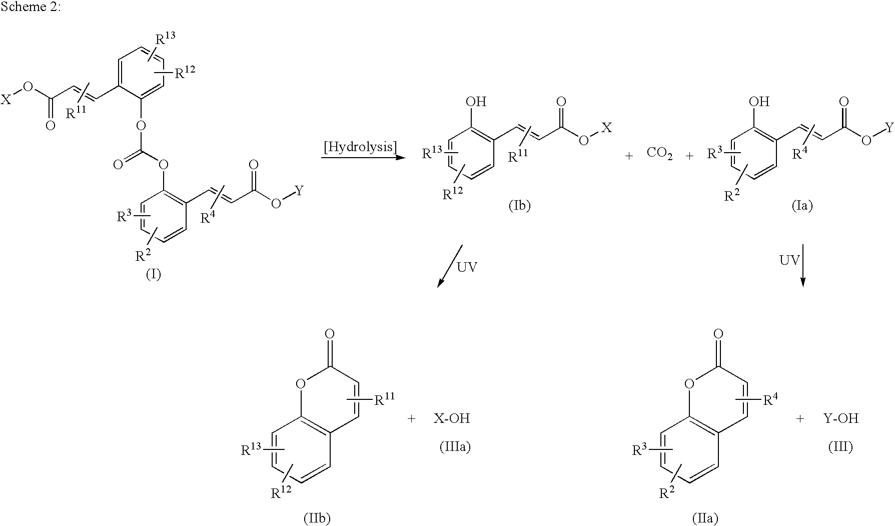

The activating conditions which lead to the first cleavage step comprise the presence of relative humidity above 20%, preferably above 30% and preferably the presence of a hydrolase such as lipase, esterase, protease or cytochrome P450.

The activating conditions which lead to the second cleavage step comprise the presence of light having a wavelength range of 200 nm to 800 nm, although irradiation with light having in its spectrum wavelengths from 250 nm to 400 nm is preferred. The release of the coumarin of formula IIa/IIIb and an alcohol YOH/XOH, or for YIX=—CR⁸═CR⁹R¹⁰ a ketone or aldehyde by tautomerisation, occurs, for example, upon exposure to sunlight penetrating through ordinary windows. Needless to say, it is upon exposure to bright sunlight, especially outdoors, that the release of these compounds will occur faster and to a greater extent than upon exposure to interior light of natural or artificial origin. The cleavage of the compound of formula Ia or Ib can also be initiated by an appropriate artificial light source, for example a sun-tanning lamp.

The compounds of formula (I) are virtually odourless and insoluble in water, i.e. the water solubility is equal to or smaller than 10 ppm.

It has been found that the use of compounds of formula (I) solves the discoloration problem. In addition, the use of compounds of the present invention also results in a very high deposition rate. Especially good results are obtainable by using Type-II compounds of the present invention. Type-II compounds of the present invention are preferred wherein R⁴═R¹¹, R³═R¹³ and R²═R¹². These molecules deposit on a fabric up to 100% by weight based on the amount added to the rinse cycle.

Most of the compounds of the present invention have never been described in literature and thus are novel in its own right.

Accordingly the present invention refers in a further aspect to a compound of formula (I)

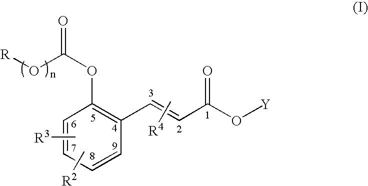

wherein the acrylic acid ester double bound is of the E configuration;

n is zero or 1;

Y is —CR⁵R⁶R⁷, wherein R⁵, R⁶ and R⁷ are independently hydrogen or a C₁-C₁₈, preferably C₁-C₁₀, hydrocarbon residue of which preferably at least one residue R⁵, R⁶ and R⁷ is not hydrogen, and the sum of all carbon atoms (R⁵+R⁶+R⁷) is not greater than 18 and at least 6, preferably the sum of all carbon atoms is between 6 and 15; or

Y is —CR⁵R⁶R⁷, wherein R⁵, R⁶ and R⁷ are independently hydrogen or a C₁-C₁₈, preferably C₁-C₁₀, aliphatic residue containing one or more atoms/groups selected from O, N and C(O), of which preferably at least one residue R⁵, R⁶ and R⁷ is not hydrogen, and the sum of all carbon atoms (R⁵+R⁶+R⁷) is not greater than 18, preferable the sum of all carbon atoms is between 6 and 15, for example Y is 2-(2-butoxy-ethoxy)-ethyl; or

Y is —CR═CR⁹R¹⁰, wherein R⁸, R⁹ and R¹⁰ are independently hydrogen or a C₁-C₁₈, preferably C₁-C₁₀, hydrocarbon residue, of which preferably at least one of the residues R⁸, R⁹ and R¹⁰ is not hydrogen, the geometry of the enol double bond is E or Z, and the sum of all carbon atoms (R⁸+R⁹+R¹⁰) is not greater than 18, preferable the sum of all carbon atoms is between 6 and 15; or

Y is —CR⁸═CR⁹R¹⁰, wherein R⁸, R⁹ and R¹⁰ are independently hydrogen or a C₁-C₁₈, preferably C₁-C₁, hydrocarbon residue containing one or more atoms/groups selected from O, N and C(O), of which preferably at least one of the residues R⁸, R⁹ and R¹⁰ is not hydrogen, the geometry of the enol double bond is E or Z, and the sum of all carbon atoms (R⁸+R⁹+R¹⁰) is not greater than 18, preferable the sum of all carbon atoms is between 6 and 15;

R² and R³ are independently hydrogen; C₁-C₆ alkyl, e.g methyl, ethyl, iso-propyl, n-butyl, tert-butyl; C₁-C₆ alkoxy residue, e.g. methoxy, ethoxy; —NO₂; —NH₂; —NHCO₂CH₃; —N(C₁-C₈ alkyl)₂, e.g. dimethylamino, diethylamino; —N(hydroxyalkyl)₂, e.g. di(hydroxyethyl)amino, di(hydroxypropyl)amino; —NHC(O)—(C₁-C₈ alkyl) or —NHC(O)—(C₃-C₈ aryl), e.g. —NHC(O)-methyl or —NHC(O)-phenyl; or

R² and R³ are attached at the positions C(6,7), C(7,8), or C(8,9), and form together with the carbon atoms to which they are attached a dioxolane ring or a dioxane ring;

R⁴ in 2- or 3-position is hydrogen; C₁-C₄ alkyl, e.g. methyl, ethyl, tert-butyl; C₂-C₄ alkenyl, e.g. vinyl, propenyl; C₃-C₆ cycloalkyl, e.g. cyclopropyl, cyclopentyl, cyclohexyl; or —CN; and

if n is zero, R is a C₂-C₂₄, preferably C₂-C₁₈, hydrocarbon residue, e.g. ethyl or phenyl; or C₁-C₂₄, preferably C₁-C₁₈, hydrocarbon residue containing one or more heteroatoms selected from N, O and Si; or

if n is 1, R is a C₁-C₂₅, preferably C₁-C₁₈, hydrocarbon residue; a C₁-C₂₅ hydrocarbon residue containing one or more atoms/groups selected from N, O, Si, and C(O); or C₁-C₂₅, preferably C₁-C₁₈, hydrocarbon residue substituted by an ionic substituent of the formula N(R²⁰)₃⁺, in which R²⁰ is the residue of an alkyl group with 1 to 18 carbon atoms, preferably 1 to 8 carbon atoms, such as trimethylammonium, or tributylammonium; or R is a monovalent residue of the formula (i)

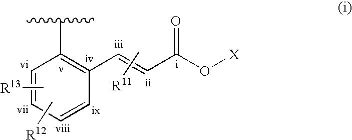

wherein
X is —CR¹⁴R¹⁵R¹⁶, wherein R¹⁴, R¹⁵ and R¹⁶ are independently hydrogen or a C₁-C₁₈, preferably C₁-C₁₀, hydrocarbon residue, of which preferably at least one residue R¹⁴, R¹⁵ and R¹⁶ is not hydrogen, and the sum of all carbon atoms (R¹⁴+R¹⁵+R¹⁶) is not greater than 18, preferable the sum of all carbon atoms is between 6 and 15; or
X is —CR¹⁴R¹⁵R¹⁶, wherein R¹⁴, R¹⁵ and R¹⁸ are independently hydrogen or a C₁-C₁₈, preferably C₁-C₁₀, hydrocarbon residue containing one or more atoms/groups selected from O, N and C(O), of which preferably at least one residue R¹⁴, R¹⁵ and
R¹⁶ is not hydrogen, and the sum of all carbon atoms (R¹⁴+R¹⁵+R¹⁶) is not greater than 18, preferable the sum of all carbon atoms is between 6 and 15, for example X is 2-(2-butoxy-ethoxy)-ethyl; or
X is —CR¹⁷=CR¹⁸R¹⁹, wherein R¹⁷, R¹⁸ and R¹⁹ are independently hydrogen or a C₁-C₁₈, preferably C₁-C₁₀, hydrocarbon residue, of which preferably at least one of the residues R¹⁷, R¹⁸ and R¹⁹ is not hydrogen, the geometry of the enol double bond is E or Z, and the sum of all carbon atoms (R¹⁷+R¹⁸+R¹⁹) is not greater than 18, preferable the sum of all carbon atoms is between 6 and 15; or
X is —CR¹⁷═CR¹⁸R¹⁹, wherein R¹⁷, R¹⁸ and R¹⁹ are independently hydrogen or a C₁-C₁₈, preferably C₁-C₁₀, hydrocarbon residue containing one or more atoms/groups selected from O, N and C(O), of which preferably at least one of the residues R¹⁷, R¹⁸ and R¹⁹ is not hydrogen, the geometry of the enol double bond is E or Z, and the sum of all carbon atoms (R¹⁷+R¹⁸+R¹⁹) is not greater than 18, preferable the sum of all carbon atoms is between 6 and 15;
R¹² and R¹³ are independently hydrogen; C₁-C₆ alkyl, e.g methyl, ethyl, iso-propyl, n-butyl, tert-butyl; C₁-C₆ alkoxy residue, e.g. methoxy, ethoxy; —NO₂; —NH₂; —NHCO₂CH₃; —N(C₁-C₆ alkyl)₂, e.g. dimethylamino, diethylamino; N(hydroxyalkyl)₂, e.g. di(hydroxyethyl)amino, di(hydroxypropyl)amino; —NHC(O)—(C₁-C₈ alkyl); or —NHC(O)—(C₃-C₈ aryl), e.g. —NHC(O)-methyl or —NHC(O)-phenyl; or
R¹² and R¹³ are attached at the positions C(vi,vii), C(vii,viii), or C(viii,ix), and form together with the carbon atoms to which they are attached a dioxolane ring or a dioxane ring;
R¹¹ in ii- or iii-position is hydrogen; C₁-C₄ alkyl, e.g. methyl, ethyl, tert-butyl; C₂-C₄ alkenyl, e.g. vinyl, propenyl; C₃-C₆ cycloalkyl, e.g. cyclopropyl, cyclopentyl, cyclohexyl; or —CN.

As used in relation to the compounds of formula (Ia) “hydrocarbon residue” unless otherwise indicated refers to aliphatic residues, e.g. alkyl, alkenyl, alkynyl, and alicyclic residues such as cycloalkyl, cycloalkenyl alkylcycloalkyl, alkenylcycloalkyl, alkenylcycloalkenyl, aryl, alkylaryl or aryfalkyl, and “hydrocarbon residues containing one or more atoms/groups selected from O, N and C(O),” refers to aliphatic residues, e.g. alkyl, alkenyl, alkynyl, and alicyclic residues such as cycloalkyl, cycloalkenyl alkylcycloalkyl, alkenylcycloalkyl, alkenylcycloalkenyl, aryl, alkylaryl or arylalkyl wherein one or more carbon atoms are replaced by O, N and/or C(O).

The compounds of formula (I) are advantageously prepared from the corresponding E-3-(2-Hydroxy-phenyl)acrylic acid esters, which in turn can be prepared for example via the following methods.

In a first step, a corresponding alcohol HO—C(R⁴R⁵R⁶) is transformed into its Li-, Na- or K-salt, preferably its Na-salt, via procedures known to the person skilled in the art, then the corresponding coumarin of formula IIa or IIb is added and the mixture is allowed to react at elevated temperature (20-120° C., preferably 50-100° C.) until complete conversion of the coumarin. Standard acid hydrolysis and workup yields the corresponding E-3-(2-Hydroxy-phenyl)acrylic acid ester, according to the general procedure described by Ganguly, N. et al; Synthetic Communications 2001, 31(2), pages 301-309.

Alternatively, salicyl aldehyde is reacted with a dialkoxyphosphoryl acetic acid ester of HO—C(R⁴R⁵R⁶), prepared via the Arbuzov reaction between the corresponding chloro- or bromoacetic acid esters and a phosphoric acid trialkyl ester, under the conditions of a Horner-reaction known to the person skilled in the art.

The preparation of coumarins of formula IIa or IIb are well known to the person skilled in the art and is described for example by A. G. Osborne et al., J. Chem. Research (S), 2003, 114-115.

The resulting E-3-(2-hydroxy-phenyl)acrylic acid ester prepared according to one of the methods described above is then acylated in a second step with an activated acid derivative, such as an acid halide or an acid anhydride, or a corresponding chloroformate under standard conditions well known to the person experienced in organic synthesis.

The compounds of formula (I) can be used in any product in which a prolonged and defined release of the abovementioned fragrant compounds is desired. Therefore, these compounds are especially useful in functional perfumery, in products which are exposed to (sun) light during or after application.

The compounds of formula (I) can act as fragrance precursors in functional and fine perfumery i.e. in fine fragrances, industrial, institutional, home and personal care products. Industrial, institutional and home cleaning products to which the compound of formula (I) can be added include all kinds of detergents, window cleaners, hard surface cleaners, all-purpose cleaners and furniture polishes. Preferably, the products are liquids, e.g. fabric conditioner compositions. A substrate, such as a fabric, treated with a product comprising a compound of formula (I) will diffuse a fresh and/or clean odor under cleavage conditions for much longer than when treated with a conventional product. Fabrics or clothes washed with such fabric softener will release the coumarins and alcohols, aldehydes or ketones even after having been stored for weeks in a dark place, e.g. a wardrobe.

The compounds of the formula (I) are also useful for application in all kinds of body care products. Especially interesting products are hair care products, for example shampoos, conditioners and hairsprays, and skin care products such as cosmetic products and especially sun protection products.

The abovementioned examples are of course only illustrative and non-limiting. Many other products to which the compounds of formula (I) may be added include soaps, bath and shower gels, deodorants and even perfumes and colognes.

The compounds of formula (I) can be used alone or in combination with other fragrance ingredients, solvents or adjuvants known to those skilled in the art. Such ingredients are described, for example, in “Perfume and Flavor Chemicals”, S. Arctander, Ed., Vol. I & II, Allured Publishing Corporation, Carol Stream, USA, 2003 and include fragrance compounds of natural or synthetic origin and essential oils.

The amounts in which the compounds of formula (I) are incorporated in the various above-mentioned products vary within a wide range. The amounts depend on the nature of the coumarines and alcohols to be released, the nature of the product to which the compounds of formula (I) are added and the desired olfactory effect. The amounts used also depend on the co-ingredients in a given composition when the compounds of formula (I) are used in admixture with perfuming co-ingredients, solvents or adjuvants. Typical concentrations are in the order of 0.01% to 5% by weight of the products.

The following non-limiting examples further illustrate the embodiments of the invention.

### EXAMPLE 1

### Preparation of 3-(2-Hydroxy-phenyl)acrylic acid 3,7-dimethyl-oct-6-enyl ester

To a suspension of NaH (114 g of a 60%-dispersion in mineral oil, 2.85 mol) in toluene (500 ml) is added at room temperature a solution of citronellol (468 g, 3.0 mol) in toluene (800 ml) over 50 min via dropping funnel. The temperature is raised to 85° C. (bath) over 60 min and stirring continued for further 45 min. Then a solution of coumarin (219 g, 1.5 mol) in toluene (800 ml) is added over 75 min. After further 90 min stirring at 80° C. (inside temperature), the deep orange suspension is cooled to 55° C. and poured on a mixture of 2.5 kg crushed ice and 280 ml 37% aq. HCl-solution. The reaction flask is rinsed twice with 500 ml toluene. Upon stirring for 10 min the colour of the organic layer fades to a pale yellow. The organic layer is washed twice with water, then with brine/water 1:1 and dried over MgSO₄. After concentrating in the rotary evaporator the excess citronellol is distilled off using a short path apparatus (110-125° C./0.03 mbar, head 82° C.) to obtain 240 g of citronellol and 463 g of a brownish residue. The latter is dissolved in 600 ml hexane containing 18 ml acetone and crystallized at −25° C. After filtration and drying 320 g (71%) of product are obtained as white crystals, m.p. 37-39° C.

IR (film): 3500-3100br, 1673vs, 1633w, 1619w, 1598s, 1450s.

¹H-NMR (400 MHz, CDCl₃): 8.10 (d, J=16, 2H), 7.79 (s, 1H), 7.44 (dd, J=7.6, 1.2, 1H), 7.22-7.18 (m, 1H), 6.69 (d, J=16, 2H), 5.09 (sym. m, 1H), 4.28 (sym. m, 24H), 2.00 (sym. m, 2H), 1.80-1.15 (series of m, 5H), 1.68 (d, J=0.4, 3H), 1.60 (s, 3H), 0.95 (d, J=6.8, 3H).

¹³C-NMR (100 MHz, CDCl₃): 169.1 (s), 155.9 (s), 141.3 (d), 131.4 (d), 131.2 (s), 129.1 (d), 124.5 (d), 121.5 (s), 120.3 (d), 117.8 (d), 116.4 (d), 63.4 (t), 36.9 (t), 35.3 (t), 29.4 (d), 25.6 (q), 25.3 (t), 19.3 (q), 17.6 (q).

MS (EI 70 eV): 302 (<1, M⁺), 165 (15), 147 (83), 138 (45), 81 (100).

### EXAMPLE 2

### Preparation of 3-(2-Hydroxy-phenyl)acrylic acid dec-9-enyl ester

To a suspension of NaH (271 g of a 60%-dispersion in mineral oil, 6.81 mol) in toluene (1200 ml) is added at room temperature a solution of 9-decen-1-ol (1060 g, 6.81 mol) in toluene (1500 ml) over 75 min via dropping funnel. After 10 min stirring a solution of coumarin (495 g, 3.39 mol) in toluene (1800 ml) is added over 75 min. The temperature is raised to 85° C. (bath) over 45 min. After further 90 min stirring at 80° C. (inside temperature), the deep orange suspension is cooled to 50° C. and poured on a mixture of 4 l 10% aq. H₂SO₄-solution and 2 l of MTBE. The organic layer is washed with saturated aq. NaHCO₃-solution, followed by water and brine. After concentrating i. RV the excess of 9-decen-1-ol is removed by thin film evaporation (130° C., 0.05 mbar) to leave 1064 g of residue which is crystallized from hexane at 5° C. From this, 775 g (75%) of product are obtained as pale yellow crystals, m.p. 58° C.

IR (film): 3197br, 1670vs, 1598vs, 1451s.

¹H-NMR (400 MHz, CDCl₃): 8.09 (d, J=16, 2H), 7.64 (s, 1H), 7.45 (d, J=8, 1H), 7.23-7.19 (m, 1H), 6.90-6.86 (m, 2H), 6.68 (d, J=16, 1H), 5.84-5.77 (sym. m, 1H), 5.01-4.91 (m, 2H), 4.23 (t, J=8, 2H), 2.03 (“q”, J=8, 2H), 1.71 (quint, J=8, 8), 1.39-1.29 (m, 1H).

¹³C-NMR (100 MHz, CDCl₃): 169.0 (s), 155.8 (s), 141.1 (d), 139.1 (d), 131.4 (d), 129.1 (d), 121.5 (s), 120.3 (d), 117.8 (d), 116.4 (d), 114.0 (t), 65.0 (t), 33.7 (t), 29.3 (t), 29.3 (t), 29.1 (t), 29.0 (t), 28.8 (t), 28.6 (t), 25.8 (t).

MS (EI 70 eV): 302 (10, M⁺), 164 (8), 178 (100), 118 (37).

### EXAMPLE 3

### 3-{2-[2-(2 Dec. 9-enyloxycarbonyl-vinylphenoxycarbonyloxy]phenyl}acrylic acid dec-9-enyl ester

To a suspension of NaH (14.9 g of a 60% dispersion in mineral oil, 0.31 mol; 1.1 equiv., oil washed away with hexane under argon atmosphere) in toluene (93 ml) is added at room temperature the solution of 9-decen-1-ol (53.2 g, 0.341 mol, 1.1 equiv.) in toluene (93 ml). The solution of coumarin (45.3 g, 0.31 mol, 1.0 equiv.) in toluene (93 ml) is then added within 30 min via dropping funnel. The temperature of the oilbath is raised slowly to 85° C. within 30 min, upon which steady evolution of hydrogen is observed. During the following 3 h stirring at 85° C. a gelatine-like orange mixture is formed which is then cooled to room temperature. The solution of phosgene in toluene (20%, 100 ml, 0.18 mol 0.6 equiv.) is added over 45 min. During the addition, the gel becomes liquid again and the reaction is cooled with an icebath. The mixture is left stirring for 16 h at room temperature, then the excess phosgene is removed by purging with Argon. The mixture is poured on 200 ml 2N aq. HCl and 200 g ice. The phases are separated and the organic layer is washed three times each with water and water/brine 1:1. After drying the over MgSO₄, the volatiles are removed i. RV and the residue dried at 0.05 mbar/50° C. for 30 min. From this, 107.1 g of product were obtained as a pale yellow oil which contains the 83% of the title compound (=90% yield) besides some mixed carbonates. A sample is further purified via column flash chromatography on SiO₂ eluting with hexane/MTBE 4:1 to isolate analytically pure product as a colourless oil.

IR (film): 1784m, 1713s, 1638m, 1202vs, 758s.

¹H-NMR (400 MHz, CDCl₃): 7.89 (d, J=16, 2H), 7.67 (dd, J=7.6, 1.2, 2H), 7.46-7.30 (m, 6H), 6.53 (d, J=16, 2H), 5.08 (sym. m, 2H), 4.26 (sym. m, 4H), 1.99 (sym. m, 4H), 1.80-1.15 (4 series of m, 10H), 1.67 (s, 6H), 1.59 (s, 6H), 0.95 (d, J=6.4, 6H).

¹³C-NMR (100 MHz, CDCl₃): 166.5 (s), 151.3 (s), 149.2 (s), 137.3 (d), 131.3 (s), 131.2 (d), 128.1 (d), 127.0 (s), 126.9 (d), 124.5 (d), 122.3 (d), 121.3 (d), 63.3 (t), 37.0 (t), 35.5 (t), 29.6 (d), 25.7 (q), 25.4 (t), 19.4 (q), 17.6 (q).

MS (EI 70 eV): 631 (5, M⁺), 493 (10), 475 (100), 431 (26), 337 (65).

### EXAMPLE 4

### 3-(2 Dec. 9-enyloxycarbonyloxy-phenyl)acrylic acid dec-9-enyl ester

### a) 3-(2-Chlorocarbonyloxy-phenyl)acrylic acid dec-9-enyl ester

The solution of 3-(2-Hydroxy-phenyl)-acrylic acid dec-9-enyl ester (prepared in Example 2, 47.1 g, 156 mmol) in toluene (550 ml) is cooled with an ice bath, then phosgene is introduced via dropping funnel (100 ml of a 20% solution, 188 mmol, 1.2 equiv), followed by N,N-diethylanilin (27.5 ml, 169 mmol). The resulting turbid solution is stirred at room temperature for 16 h, after which the excess phosgene is removed by extensive purging with argon. The white suspension is poured on a mixture of 2 N aq. HCl-solution and ice, the phases are separated and the aqueous layer is extracted further with toluene. The organic layers are washed with 2 N aq. HCl-solution, then 3 times with brine and combined. After drying over MgSO₄, the solvent is removed and the residue dried at 0.05 mbar/50° C. for 20 min. From this 57.2 g (100%) of product are obtained as a pale yellow oil which is not further purified.

IR (film): 1786s, 1714s, 1639m, 1171s, 1108vs.

¹H-NMR (400 MHz, CDCl₃): 7.77 (d, J=16, 1H), 7.66 (d, J=8, 1H), 7.44 (m, 1H), 7.35 (t, J=7, 1H), 7.27 (d, J=8, 1H), 6.50 (d, J=16, 1H), 5.81 (m, 1H), 4.99 (dd, J=17, 1, 1H), 4.93 (dd, J=10, 1, 1H), 4.22 (t, J=7, 2H), 2.04 (q, J=7, 2H), 1.71 (qd, J=7, 7,2H), 1.36 (br. m, 10H).

¹³C-NMR (100 MHz, CDCl₃): 166.2 (s), 149.6 (s), 149.2 (s), 139.0 (d), 136.4 (d), 131.2 (d), 128.1 (d), 127.6 (d), 126.6 (s), 121.8 (d), 121.8 (d), 114.1 (t), 64.9 (t), 33.7 (t), 29.3 (t), 29.1 (t), 28.9 (t), 28.8 (t), 28.6 (t), 25.8 (t).

MS (EI 70 eV). 344 (4, M⁺), 227 (6), 209 (50), 181 (15), 147 (100), 118 (29).

### b) 3-(2 Dec. 9-enyloxycarbonyloxy-phenyl)acrylic acid dec-9-enyl ester

The chloroformate prepared above (3.65 g, 10 mmol) in toluene (10 ml) is added dropwise to the solution of 9-decen-1-ol (1.56 g, 10 mmol) and pyridine (2.0 ml, 25 mmol) in toluene (20 ml) with external ice cooling. The resulting white suspension is warmed to room temperature and stirred for 4 h, then 2 N aq. HCl-solution (20 ml) is added and stirring continued for 5 min. The mixture is then transferred into a separatory funnel, the phases are separated and the aqueous phase is further extracted with MTBE. The organic layers are washed with brine/H₂O 1:1 and dried over MgSO₄. Removal of the solvents and drying at 0.05 mbar (at room temperature) yields a crude which is purified via flash chromatography on SiO₂ eluting with MTBE/hexane 4:1. From this, 3.89 g (80%) of product are obtained as a colourless viscous oil.

IR (film): 2926m, 1764s, 1715s, 1639w, 1212vs.

¹H-NMR (400 MHz, CDCl₃): 7.82 (d, J=16, 1H), 7.62 (dd, J=7.6/1.2, 1H), 7.40 (br. t, J=8, 1H), 7.29-7.20 (m, 2H), 6.48 (d, J=16, 1H), 5.85-5.78 (m, 2H), 5.02-4.92 (m, 4H), 4.26 (t, 6.8, 2H), 4.19 (t, J=6.8, 2H), 2.07-2.02 (m, 4H), 1.80-1.68 (m, 4H), 1.38-1.32 (m, 20H).

¹³C-NMR (100 MHz, CDCl₃): 166.6 (s), 153.2 (s), 149.4 (s), 139.0 (d), 139.0 (d), 137.6 (d), 130.9 (d), 127.7 (d), 127.03 (s), 126.3 (d), 122.5 (d), 120.6 (d), 114.0 (t), 114.0 (t), 69.2 (t), 64.6 (t), 33.6 (2 t), 29.2 (t), 29.2 (t), 29.1 (t), 29.0 (t), 29.8 (t), 28.9 (t), 28.8 (t), 28.6 (t), 28.4 (t), 25.8 (t), 25.5 (t).

MS (EI 70 eV) 484 (3, M⁺), 328 (3), 302 (3), 164 (14), 146 (100), 118 (20).

### EXAMPLE 5

### 3-[2-(3,7-Dimethyl-oct-6-enyloxycarbonyloxy)phenyl]acrylic acid 3,7-dimethyl-oct-6-enyl ester

The solution of 3-(2-Hydroxy-phenyl)acrylic acid 3,7-dimethyl-oct-6-enyl ester (6.04 g 20 mmol, as prepared in Example 1) and pyridine (4.0 ml, 50 mmol) in toluene (40 ml) is cooled with an icebath and the solution of citronellyl chloroformate (5.06 g, 22 mmol) in toluene (20 ml) is added dropwise via dropping funnel over 20 min. The resulting suspension is stirred at room temperature during 3 days, then quenched by addition of 2 N aq. HCl-solution at 2-3° C. Extraction, separation of phases, washing of the organic layer with brine and drying over MgSO₄, followed by removal of the solvents and drying of the residue under high vacuum yielded 9.7 g (100%) of analytically pure product as a pale yellow oil.

IR (film):1765m, 1715m, 1638w, 1213vs.

¹H-NMR (400 MHz, CDCl₃): 7.81 (d, J=16, 1H), 7.62 (dd, J=1.2 and 7.6, 1H), 7.29-7.20 (m, 2H), 6.47 (d, J=16, 1H), 5.10 (sym. m, 2H), 4.32-4.22 (m, 4H), 2.00 (sym. m, 4H), 1.85-1.15 (series of m, 10H), 1.68 (“s”, 6H), 1.59 (s, 6H), 0.96 (d, J=6, 3H), 0.94 (d, J=6, 3H).

¹³C-NMR (100 MHz, CDCl₃): 166.5 (s), 153.2 (s), 149.4 (s), 137.6 (d), 131.3 (s), 131.2 (s), 130.9 (d), 127.8 (d), 127.0 (s), 126.3 (d), 124.4 (d), 124.3 (d), 122.5 (d), 120.6 (d), 67.7 (t), 63.1 (t), 36.9 (t), 36.8 (t), 35.4 (t), 35.2 (t), 29.5 (d), 29.2 (d), 25.6 (q), 25.3 (t), 25.2 (t), 19.3 (q), 19.2 (q), 17.5 (q).

MS (APCI with NH₄OAc, pos.): 502 ([M+NH₄]⁺), 485 (12, M⁺).

### EXAMPLE 6

### 3-(2-Hex-3-enyloxycarbonyloxy-phenyl)acrylic acid 3,7-dimethyl-oct-6-enyl ester

Repeating the procedure of Example 5 with 3-(2-Hydroxy-phenyl)acrylic acid 3,7-dimethyl-oct-6-enyl ester (6.04 g 20 mmol), pyridine (4.0 ml, 50 mmol) and cis-3-hexenyl chloroformate (3.58 g, 22 mmol) yields 8.60 g (100%) analytically pure product as a pale yellow oil.

IR (film): 1765m, 1715m, 1638w, 1212vs.

¹H-NMR (400 MHz, CDCl₃): 7.81 (d, J=16, 1H), 7.62 (dd, J=1.2 and 7.6, 1H), 7.39 (sym. m, 1H), 7.29-7.20 (m, 2), 6.47 (d, J=16, 1H), 5.56 (sym. m, 1H), 5.36 (sym. m, 1H), 5.10 (sym. m, 1H), 4.27-4.23 (m, 4H), 2.52 (q, J=8, 4H), 2.11-1.90 (m, 4H), 1.80-1.15 (series of m, 5H), 1.68 (s, 3H), 1.61 (s, 3H), 0.97 (t, J=8, 3H), 0.95 (d, J=8, 3H).

¹³C-NMR (100 MHz, CDCl₃): 166.5 (s), 153.2 (s), 149.4 (s), 137.5 (d), 135.2 (d), 131.2 (s), 130.9 (d), 127.8 (d), 127.0 (s), 126.3 (d), 124.4 (d), 122.5 (d), 122.5 (d), 120.6 (d), 68.4 (t), 63.1 (t), 36.9 (t), 35.4 (t), 29.5 (d), 26.5 (t), 25.6 (q), 25.3 (t), 19.3 (q), 17.5 (q), 14.0 (q).

MS (APCI with NH₄OAc, pos.): 446 ([M+NH₄]⁺), 429 (11, M⁺).

### EXAMPLE 7

### 3-(2-Acetoxy-phenyl)acrylic acid dec-9-enyl ester

3-(2-Hydroxy-phenyl)acrylic acid dec-9-enyl ester (6.07 g, 20 mmol, as prepared in Example 2) in toluene (40 ml) are added to a suspension of NaH (840 mg of a 60% dispersion in mineral oil, 20 mmol, 1 equiv.) in toluene (60 ml). The yellow suspension is stirred for 30 min. at RT, then acetyl chloride (1.96 g, 25 mmol, 1.25 equiv.) are added. The resulting colourless solution is stirred for another 60 min. at RT, then poured on H₂O (100 ml). The organic layer is separated, and the aqueous layer extracted with MTBE. The organic layers are washed with 1 N aq. NaHCO₃-solution, then water and brine. After drying over MgSO₄, the solvents are removed to yield 6.85 g (99%) of product as a colourless oil.

IR (film): 1770m, 1713s, 1638w, 1170vs.

¹H NMR (400 MHz, CDCl₃): 8 ppm 1.34 (m, 9H), 1.68 (qd, J=7, 7, 2H), 2.03 (q, J=7, 2H), 2.34 (s, 3H), 4.18 (t, J=7, 2H), 4.93 (m, 2H), 5.80 (m, 1H), 6.45 (d, J=16, 1H), 7.10 (m, 1H), 7.22 (t, J=7.58, 1H), 7.36 (m, 1H), 7.62 (dd, J=8, 2, 1H), 7.75 (d, J=16, 1H).

MS (EI 70 eV): 344 (3, M⁺), 302 (18), 285 (10), 164 (12), 146 (100), 136 (11), 118 (30).

### EXAMPLE 8

### 3-(2-Hydroxy-phenyl)-acrylic acid phenethyl ester

The compound is prepared by reaction of coumarin with phenylethanol in the presence of sodium hydride following the procedure described in Example 2. The title compound is isolated as a white solid, m.p. 40° C.

IR (film): 3261 vs, 1771m, 1738s, 1679vs, 1631vs, 1599 vs, 1174 vs, 752 vs, 700 vs.

¹³C-NMR (CDCl₃, 100 MHz): 168.6 (s), 155.8 (s), 141.3 (d), 137.7 (d), 131.5 (d), 129.1 (d), 128.9 (d), 128.5 (d), 126.5 (d), 121.5 (s), 120.4 (d), 117.7 (d), 116.4 (d), 65.2 (t), 35.1 (t).

¹H-NMR (CDCl₃, 400 MHz): 8.07 (d, J=16, 1H), 7.49 (s, 1H), 7.42 (dd, J=8, 2, 1H), 7.27-7.32 (m, 2H), 7.17-7.26 (m, 5H), 6.86 (td, J=8, 1.1, 2H), 6.64 (d, J=16, 1H), 4.43 (t, J=7, 2H), 3.01 (t, J=7, 2H).

MS (EI, 70 eV): 268 (<1, M⁺), 266 (1), 250 (4), 164 (46), 146 (69), 118 (60), 104 (79), 91 (100).

### EXAMPLE 9

### 3-(2-Phenethyloxycarbonyloxy-phenyl)-acrylic acid phenethyl ester

A solution of 3-(2-Hydroxy-phenyl)-acrylic acid phenethyl ester (1.61 g, 6.0 mmol) in toluene (15 ml) is added dropwise at room temperature to a suspension formed from chloroformic acid phenethyl ester (1.22 g, 6.6 mmol, 1.1 equiv.) and pyridine (0.97 ml, 12.0 mmol, 2.0 equiv.) in toluene (10 ml). The mixture is further stirred at room temperature for 16 h, then worked up following the genera procedure described for Example 5 and purified by flash chromatography on SiO₂, eluting with hexane/MTBE 4:1. The title compound (1.61 g, 64% yield) is obtained as a colourless, viscous oil.

IR (film): 1760s, 1710 s, 1212 vs, 1168 vs, 697 vs.

¹H-NMR (CDCl₃, 400 MHz): 7.82 (d, J=16.2 Hz, 1H), 7.60 (dd, J=7.8, 1.6 Hz, 1H), 7.36-7.43 (m, 1H), 7.20-7.35 (m, 11H), 7.16 (dd, J=8.2, 1.1 Hz, 1H), 6.46 (d, J=16.2 Hz, 1H), 4.37-4.51 (m, 4H), 2.97-3.10 (m, 4H).

¹³C-NMR (CDCl₃, 100 MHz): 166.5 (s), 153.2 (s), 149.5 (s), 137.9 (s), 137.8 (s), 136.8 (d), 131.1 (d), 129.7 (d), 129.0 (d), 128.9 (d), 128.9 (d), 128.6 (d), 128.6 (d), 128.5 (d), 127.8 (d), 127.8 (d), 127.0 (s), 126.8 (d), 126.6 (d), 126.5 (d), 122.6 (d), 120.5 (d), 69.4 (t), 65.1 (t), 35.2 (t), 35.0 (t).

MS (EI, 70 eV): 417 (<1, [M+1 ]⁺), 370 (4), 164 (8), 146 (7), 105 (100), 104 (31).

### EXAMPLE 10

### 3-[2-(3-Methyl-5-phenyl-pentyloxycarbonyloxy)-phenyl]-acrylic acid phen-ethyl ester

Following the general procedure described in Example 9 the title compound is prepared, using 3-(2-hydroxy-phenyl)-acrylic acid phenethyl ester (1.61 g, 6.0 mmol), chloroformic acid 3-methyl-5-phenyl-pentyl ester (1.59 g, 6.6 mmol, 1.1 equiv.) and pyridine (0.97 ml, 12.0 mmol, 2.0 equiv.). 3-[2-(3-Methyl-5-phenyl-pentyloxycarbonyloxy)-phenyl]-acrylic acid phenethyl ester (1.65 g, 58% yield) is obtained as colourless, viscous oil.

IR (film):1760 s, 1712 s, 1251 s, 1211 vs, 1167 vs, 697 vs.

¹H-NMR (CDCl₃, 400 MHz): 7.82 (d, J=16.2 Hz, 1H), 7.61 (dd, J=7.8, 1.6 Hz, 1H), 7.37-7.44 (m, 1H), 7.13-7.35 (m, 13H), 6.46 (d, J=16.2 Hz, 1H), 4.41 (t, J=7.1 Hz, 2H), 4.25-4.37 (m, 2H), 3.00 (t, J=7.1 Hz, 2H), 1.79-1.94 (m, 1H), 1.46-1.76 (m, 5H), 1.02 (d, J=6.3 Hz, 3H)

¹³C-NMR (CDCl₃, 100 MHz): 166.5 (s), 153.3 (s), 149.5 (s), 142.5 (s), 137.9 (d), 137.8 (s), 131.1 (d), 128.9 (d), 128.5 (d), 128.3 (d), 128.3 (d), 127.9 (d), 127.0 (s), 126.6 (d), 126.5 (d), 125.7 (d), 122.6 (d), 120.5 (d), 67.7 (t), 65.1 (t), 38.8 (t), 35.3 (t), 35.2 (t), 33.2 (t), 29.4 (d), 19.4 (q).

MS (EI, 70 eV): 472 (<1, M⁺), 426 (4), 313 (5), 164 (34), 160 (39), 146 (32), 105 (91), 91 (100).

### EXAMPLE 11-15

Further compounds as listed in Table 1 were prepared according to the general procedure described in the specification.

TABLE 1

| | | |
|---|---|---|
| IR (film) | MS (EI 70 ev) | NMR (¹H NMR (400 MHz, CDCl₃) δ in ppm) |

Benzoic acid 4-benzoylamino-2-[2-(3,7-dimethyl-oct-6-enyloxycarbonyl)vinyl]phenyl
ester (11)

| | | |
|---|---|---|
| 1713s, | 527 ([M + 2]⁺), | 0.87 (m, 3H), 1.15 (m, 1H), 1.31 (m, 2H), 1.44 (m, 2H), |
| 1654s, | 388 (26), | 1.52 (m, 2H), 1.59 (s, 3H), 1.66 (s, 3H), 1.94 (m, 2H), 4.17 (m, |
| 1525s, | 371 (24), 265 (33), | 2H), 5.08 (m, 1H), 6.52 (d, J = 16, 1H), 7.25 (d, J = 9, 1H), |
| 1196vs, | 105 (100). | 7.55 (m, 5H), 7.66 (m, 2H), 7.80, (d, J = 16, 1H), 7.90 (m, 2H), |
| 704vs. | | 8.13 (d, J = 3, 1H), 8.23 (dd, J = 8, 1, 2H). |

3-(2-Acetoxy-5-tert-butyl-phenyl)acrylic acid 3,7-dimethyl-oct-6-enyl ester (12)

| | | |
|---|---|---|
| 1768m, | 400 (2, M⁺), | 0.94 (d, J = 7, 3H), 1.18-1.80 (series of m, 5H), 1.33 (s, 9H), |
| 1713s, | 262 (29, 220 | 1.61 (s, 3H), 1.68 (s, 3H), 2.0 (m, 2H), 2.36 (s, 3H), 4.25 (m, |
| 1182vs. | (28), 202 (66), | 2H), 5.10 (m, 1H), 6.45 (d, J = 16, 1H), 7.04 (d, J = 9, 1H), |
| | 187 (100), | 7.42 (dd, J = 9, 2, 1H), 7.62 (d, J = 2, 1H), 7.74 (d, J = 16, 1H). |
| | 159 (30). | |

3-(2-Acetoxy-phenyl)acrylic acid 3,7-dimethyl-oct-6-enyl ester (13)

| | | |
|---|---|---|
| 1770m, | 344 (<1, M⁺), | 0.95 (d, J = 7, 3H), 1.23 (ddd, J = 9, 7, 6, 1H), 1.37 (m, 1H), |
| 1713s, | 147 (92), | 1.51 (m, 1H), 1.61 (s, 3H), 1.58-1.80 (m, 2H), 1.68 (s, 3H), |
| 1637w, | 138 (68), 95 (76), | 2.01 (m, 2H), 2.37 (s, 3H), 4.24 (m, 2H), 5.10 (ddd, J = 7, 6, |
| 1174vs. | 81 (100). | 1, 1H), 6.45 (d, J = 16, 1H), 7.12 (dd, J = 8, 1, 1H), 7.26 (m, |
| | | 1H), 7.40 (td, J = 8, 2, 1H), 7.64 (dd, J = 8, 2, 1H), 7.74 (d, |
| | | J = 16, 1H). |

3-(2-Ethoxycarbonyloxy-phenyl)acrylic acid dec-9-enyl ester (14)

| | | |
|---|---|---|
| 1763s, | 374 (4, M⁺), | 1.37 (m, 13H), 1.69 (qd, J = 7, 7, 2H), 2.04 (q, J = 7, 2H), |
| 1714s, | 285 (4), | 4.19 (t, J = 7, 2H), 4.33 (q, J = 7, 2H), 4.93 (d, J = 10, 1H), |
| 1638m, | 192 (3), 164 (7), | 4.99 (dd, J = 17, 2, 1H), 5.81 (m, 1H), 6.48 (d, J = 16, 1H), |
| 1249s, | 146 (100), | 7.25 (m, 2H), 7.40 (m, 1H), 7.63 (d, J = 8, 1H), 7.82 (d, |
| 1212vs, | 118 (34). | J = 16, 1H). |
| 1170vs | | |

3-(2-{2-[2-(3,7-Dimethyl-oct-6-enyloxycarbonyl)vinyl]phenoxycarbonyloxy}phenyl)-
acrylic acid 3,7-dimethyl-oct-6-enyl ester (15)

| | | |
|---|---|---|
| 1784w, | 630 (4, M⁺), | 0.95 (d, J = 6, 6H), 1.18-1.80 (series of m, 10H), 1.57 (s, 6H), |
| 1712s, | 493 (12), | 1.67 (d, J = 1, 6H), 1.99 (m, 4H), 4.26 (m, 4H), |
| 1638w, | 475 (100), | 5.08 (m, 2H), 6.53 (d, J = 16, 2H), 7.31 (m, 4H), 7.44 (m, 2H), |
| 1165vs. | 431 (15), 337 (65), | 7.65 (dd, J = 8, 2, 2H), 7.90 (d, J = 16, 2H). |
| | 249 (60), | |
| | 147 (60). | |

### EXAMPLE 16

### 3-(2-Hydroxy-4-methoxy-phenyl)-acrylic acid 3,7-dimethyl-oct-6-enyl ester

The compound is prepared by reaction of 7-methoxycoumarin with citronellol in the presence of sodium hydride following the procedure described in Example 2. The title compound is isolated as a viscous, colourless oil.

IR (film):3331 br., 1705 s, 1676 s, 1611 vs, 1518 m, 1169 vs, 836 w, 802 w.

¹H-NMR (CDCl₃, 400 MHz): 7.98 (d, J=16.2 Hz, 1H), 7.30-7.39 (m, 2H), 6.34-6.63 (m, 3H), 5.03-5.20 (m, 1H), 4.18-4.37 (m, 2H), 3.79 (s, 3H), 2.06 (m, 2H), 1.10-1.87 (m, 5H), 1.68 (s, 3H), 1.61 (s, 3H), 0.95 (d, J=6.6 Hz, 3H).

MS (EI, 70 eV): 348 (<1, M⁺), 194 (81), 177 (87), 176 (100), 148 (54), 133 (39).

### EXAMPLE 17

### 3-(2-Hydroxy-5-methyl-phenyl)-acrylic acid 2-ethyl-4-(2,2,3-trimethyl-cyclopent-3-enyl)-but-2-enyl ester

The compound is prepared by reaction of 6-methylcoumarin with 2-ethyl-4-(2,2,3-trimethyl-cyclopent-3-enyl)-but-2-en-1-ol in the presence of sodium hydride following the procedure described in Example 2. The compound is isolated as a viscous, slightly yellow oil.

IR (film):3313 s, 3035m, 2958 vs, 1688 s, 1627 s, 1508 m, 1463 s, 1155 s, 816 m, 799 m.

¹H-NMR (CDCl₃, 200 MHz 8.00 (d, J=16.2 Hz, 1H), 7.17-7.36 (m, 1H), 6.92-7.13 (m, 2H), 6.74 (d, J=8.2 Hz, 1H), 6.61 (d, J=16.2 Hz, 1H), 5.31-5.63 (m, 2H), 5.22 (s, 2H), 4.66 (s, 1H), 4.07 (s, 2H), 2.26 (s, 3H), 1.67-2.45 (m, 4H), 1.62-1.56 (m, 3H), 0.90 (t, J=7.6 Hz, 3H), 0.89 (s, 3H), 0.78 (s, 3H).

MS (EI, 70 eV): 368 (<1, M⁺), 190 (17), 161 (53), 108 (100).

### EXAMPLE 18

### 3-(2-Hydroxy-4-methoxy-phenyl)-acrylic acid 3-methyl-5-phenyl-pentyl ester

The compound is prepared by reaction of 7-methoxycoumarin with citronellol in the presence of sodium hydride following the procedure described in Example 2. The compound is isolated as a white solid, m.p. 70-73° C.

IR (film):1760 s, 1712 s, 1251 s, 1211 vs, 1167 vs, 697 vs.

¹H-NMR (CDCl₃, 400 MHz): 8.00 (d, 4=15.9 Hz, 1H), 7.93 (br. s, 1H), 7.11-7.39 (m, 6H), 6.55 (d, J=15.9 Hz, 1H), 6.42-6.49 (m, 2H), 4.19-4.34 (m, 2H), 3.74 (s, 3H), 2.52-2.74 (m, 2H), 1.41-1.89 (m, 5H), 1.00 (d, J=6.3 Hz, 3H).

¹³C-NMR (CDCl₃, 100 MHz): 169.4 (s), 162.5 (s), 157.5 (s), 142.6 (s), 141.1 (s), 130.5 (s), 128.3 (d), 125.6 (d), 115.0 (s), 114.9 (d), 106.8 (d), 101.7 (d), 63.0 (t), 55.3 (q), 38.7 (t), 35.4 (t), 33.2 (t), 29.5 (d), 19.4 (q).

MS (EI, 70 eV). 354 (30, M⁺), 194 (78), 176 (100), 148 (46), 133 (17), 104 (14), 91 (40).

### EXAMPLES 19

The compounds of Example 16, 17 and 18 may be used as intermediates for the preparation of further compounds A, B and C according to the present invention. Their preparation may be carried with the corresponding chloroformates following the general procedure of Example 9.

| | |
|---|---|
| 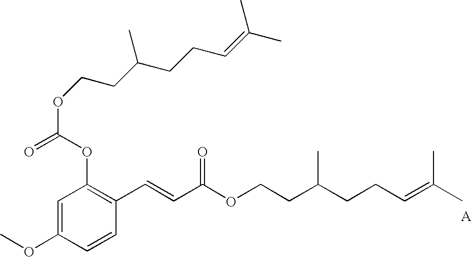 | 3-[2-(3,7-Dimethyl-oct-6-enyloxycarbonyloxy)-4-methoxy-phenyl]-acrylic acid 3,7-dimethyl-oct-6-enyl ester |
| 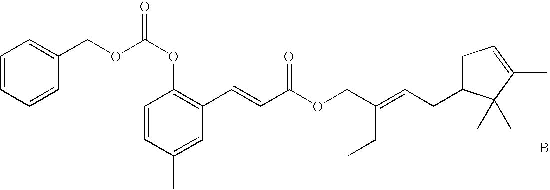 | 3-(2-Benzyloxycarbonyloxy-5-methyl-phenyl)-acrylic acid 2-ethyl-4-(2,2,3-trimethyl-cyclopent-3-enyl)-but-2-enylester |
| 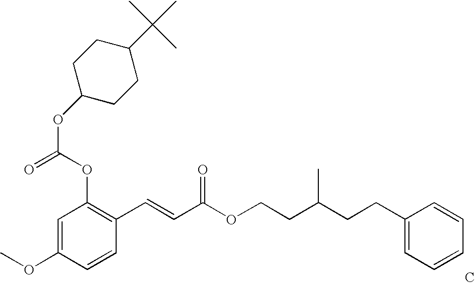 | 3-[2-(4-tert-Butyl-cyclohexyloxycarbonyloxy)-4-methoxy-phenyl]-acrylic acid 3-methyl-5-phenyl-pentyl ester |

### EXAMPLE 20

### UV-Spectra Comparison (Protected/Non Protected Fragrance in Presence of a Fabric Conditioner)

Solutions A to D, 5 ml each, were prepared using CH₃CN/H₂O (3:2) as a solvent and their colour visually judged.

| | Fragrance Precursor | Additive | |
|---|---|---|---|
| Solution | (each added at 0.1 mg/ml) | (amount) | Colour |
| A | (E)-3-(2-Hydroxy-phenyl)acrylic acid | none | colourless |
| | dec-9-enyl ester (Ex. 2) | | |
| B | As above (A) | Fabric | Bright |
| | | conditioner¹⁾ | yellow |
| | | (30 mg) | |
| C | 3-{2-[2-(2-Dec-9-enyloxycarbonyl- | none | colourless |
| | vinyl)phenoxycarbonyloxy]-phenyl}- | | |
| | acrylic acid dec-9-enyl ester (Ex. 3) | | |
| D | As above (C) | Fabric | colourless |
| | | conditioner¹⁾ | |
| | | (30 mg) | |

¹⁾Aqueous fabric conditioner emulsion containing 12.7% wt/wt of active cationic surfactant

After adding a fabric conditioner to a solution comprising an unprotected fragrance precursor (solution A/B) the solution turned bright yellow, whereas a solution comprising a protected fragrance precursor (solution C/D) remains colourless.

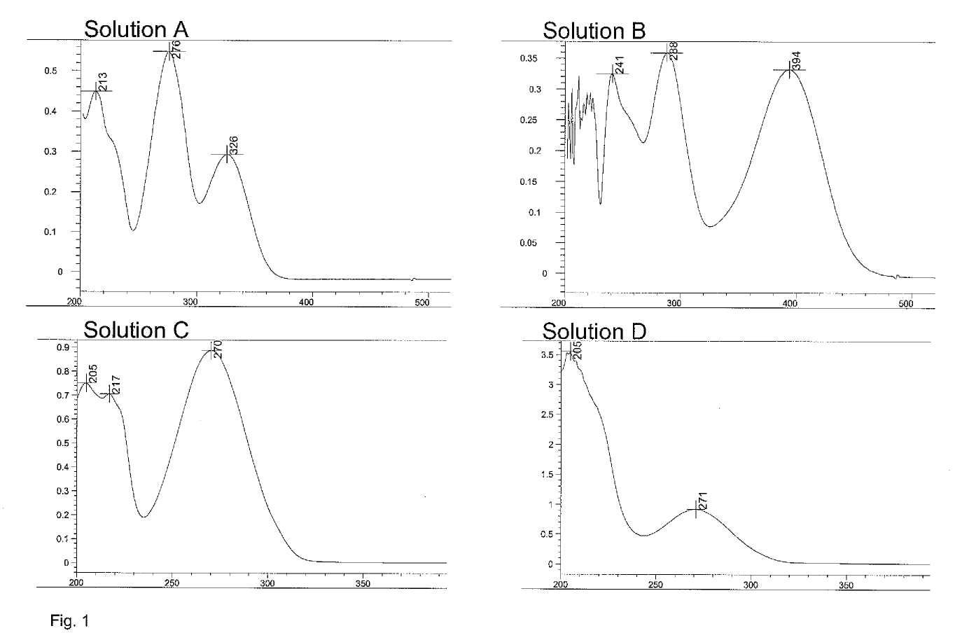

The UV-spectra of solutions A to D were recorded and are depicted in FIG. 1. They show that upon addition of the fabric conditioner the highest λₘₐₓ at 326 nm (UV) shifts to 394 nm (visible light), whereas the highest πₘₐₓ of the protected fragrance precursor remains unchanged at 270 nm upon addition of the fabric conditioner.

### EXAMPLE 21

### Application in Fabric Conditioner

The deposition and cleavage of the compounds of formula (I) on cotton (white towels) in a typical wash/rinse cycle is determined as described in the following. All handling of samples containing a compound of formula (I) is done with as little exposure to light as possible.

An aqueous fabric conditioner emulsion containing 12.7% wt/wt of active cationic surfactant and 0.5% wt/wt of a compound of formula (I) is prepared. Whereas samples with free o-coumarates turn yellow, samples with protected o-coumarates remain white. A wash/rinse cycle (40° C. program) in a standard washing machine is performed with a 1 kg wash load consisting of 25 cotton terry towels, adding the following:
1) 62 g of a standard concentrated unperfumed washing powder containing protease, cellulase, and lipase for the washing cycle
2) 16 g of fabric conditioner as prepared above for the rinse cycle (containing 80 mg of a compound of formula (I) as indicated in Table 2, compound Ex. No 6, 5, 3, 4 or 14, or a compound of formula (A), i.e. comp. Ex. No. 1 or 2, as comparison as indicated in Table 2.

The towels are removed from the washing machine and dried in the dark for 24 h at room temperature and 40% relative humidity. Three towels (representing each 4% of the total wash load) are removed and placed individually in a Soxhlet apparatus containing 0.5 l of methylene chloride and extracted for 5 h. The solvent is removed carefully in a rotary evaporator and the residues are standardized to 10 ml acetonitrile solution. These solutions are analyzed by RP-HPLC using a H₂O/acetonitrile gradient and UV-detection at 258 nm. The concentrations of “protected” and “unprotected” fragrance precursor per sample are determined via external calibration and a mean value is calculated from the three towels. From this, the mean deposition rate in % of theory compared to the molar amount of protected precursor applied via the fabric conditioner for the two precursor types is calculated. The results are listed in Table 2.

Best cleavage rate by lipase is observed for carbonates derived from short chain primary alcohols (Comp. Ex. No. 6) and (Comp. Ex. No 14). The best combined deposition/cleavage result is obtained from the symmetrical carbonate (Comp. Ex. No. 3). The final deposition of free precursor using this compound is higher than by administration of the corresponding “unprotected” 3-(2-Hydroxy-phenyl)-acrylic acid ester (Comp. Ex. No. 2).

As used herein “unprotected fragrance precursor” means a compound comprising a hydroxyl group, i.e. the prior art compound (A), and “protected fragrance precursor” means a compound of formula (I) according to the present invention.

TABLE 2

| | | | Releases | Modified | | |
|---|---|---|---|---|---|---|
| Comp. | | Protecting | Coumarin | Prec. | Free prec. | Cleavage |
| Ex. N^{o} | Fragrance precursor | group | and | [Depos. %] | [Depos. %] | [%] |
| 1 | 3-(2-Hydroxy-phenyl)acrylic acid 3,7- | None | Citronellol | — | 45 | — |
| | dimethyl-oct-6-enyl ester | | | | | |
| 2 | 3-(2-Hydroxy-phenyl)acrylic acid dec-9- | None | 9-Decen-1-ol | — | 46 | — |
| | enyl ester | | | | | |
| 6 | 3-(2-Hex-3-enyloxycarbonyloxy- | cis-3-hex. carbonate | Citronellol & | 2 | 50 | 97 |
| | phenyl)acrylic acid 3,7-dimethyl-oct-6- | | cis-3-hexenol | | | |
| 5 | 3-[2-(3,7-Dimethyl-oct-6- | Citronellylcarbonate | Citronellol | 69 | 11 | 14 |
| | enyloxycarbonyloxy)-phenyl]acrylic | | | | | |
| | acid 3,7-dimethyl-oct-6-enyl ester | | | | | |
| 3 | 3-{2-[2-(2-Dec-9-enyloxycarbonyl- | Sym. Carbonate | 9-Decen-1-ol | 34 | 66 | 66 |
| | vinyl)phenoxycarbonyloxy]phenyl}- | | | | | |
| | acrylic acid dec-9-enyl ester | | | | | |
| 4 | 3-(2-Dec-9-enyloxycarbonyloxy- | Rosalva-Carbonate | 9-Decen-1-ol | 32 | 34 | 68 |
| | phenyl)acrylic acid dec-9-enyl ester | | | | | |
| 14 | 3-(2-Ethoxycarbonyloxy-phenyl)acrylic | Ethylcarbonate | 9-Decen-1-ol | 5 | 42 | 91 |
| | acid dec-9-enyl ester | | | | | |

1. A process for preparing compositions which provide upon activation an olfactory compound comprising incorporating into the composition a compound of formula (I) as precursor for olfactory compounds compound 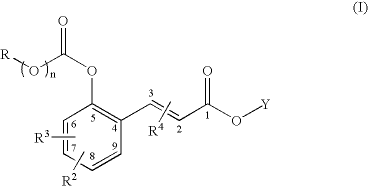
wherein the acrylic acid ester double bound is of the E configuration;
n is zero or 1;
Y is —CR⁵R⁶R⁷, wherein R⁵, R⁶ and R⁷ are independently hydrogen or a C₁-C₁₈ hydrocarbon residue, and the sum of all carbon atoms (R⁵+R⁶+R⁷) is not greater than 18; or
Y is —CR⁵R⁶R⁷, wherein R⁵, R⁶ and R⁷ are independently hydrogen or a C₁-C₁₈ hydrocarbon residue containing one or more atoms/groups selected from O, N and C(O), and the sum of all carbon atoms (R⁵+R⁶+R⁷) is not greater than 18; or
Y is —CR⁸═CR⁹R¹⁰, wherein R⁸, R⁹ and R¹⁰ are independently hydrogen or a C₁-C₁₈ hydrocarbon residue, the geometry of the enol double bond is E or Z, and the sum of all carbon atoms (R⁸+R⁹+R¹⁰) is not greater than 18; or
Y is CR⁸═CR⁹R¹⁰, wherein R⁸, R⁹ and R¹⁰ are independently hydrogen or a C₁-C₁₈ hydrocarbon residue containing one or more atoms/groups selected from O, N and C(O), the geometry of the enol double bond is E or Z, and the sum of all carbon atoms (R⁸+R⁹+R¹⁰) is not greater than 18;
R² and R³ are independently hydrogen, C₁-C₆ alkyl, C₁-C₆ alkoxy residue, —NH₂, —NO₂, —NHCO₂CH₃, —N(C₁-C₆ alkyl)₂, —N(hydroxyalkyl)₂, —NHC(O)—(C₁-C₈ alkyl) or —NHC(O)—(C₃-C₈ aryl); or
R² and R³ are attached at the positions C(6,7), C(7,8), or C(8,9), and form together with the carbon atoms to which they are attached a dioxolane ring or a dioxane ring;
R⁴ in 2- or 3-position is hydrogen, C₁-C₄ alkyl, C₂-C₄ alkenyl, C₃-C₆ cycloalkyl, or —CN; and
a) if n is zero, R is a C₁-C₂₄ hydrocarbon residue, or C₁-C₂₄ hydrocarbon residue containing one or more heteroatoms selected from N, O and Si; or
b) if n is 1, R is a C₁-C₂₅ hydrocarbon residue, a C₁-C₂₋₅ hydrocarbon residue containing one or more atoms/groups selected from N, O, Si, and C(O), or C₁-C₂₅ hydrocarbon residue substituted by an ionic substituent of the formula N(R²⁰)₃⁺, in which R²⁰ is the residue of an alkyl group with 1 to 18 carbon atoms; or
R is a monovalent residue of the formula (I) 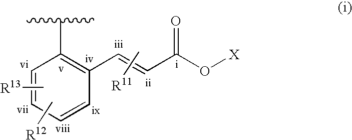
wherein
X is —CR¹⁴R¹⁵R¹⁶, wherein R¹⁴, R¹⁵ and R¹⁶ are independently hydrogen or a C₁-C₁₈ hydrocarbon residue, and the sum of all carbon atoms (R¹⁴+R¹⁵+R¹⁶) is not greater than 18; or
X is —CR¹⁴R¹⁵R¹⁶, wherein R¹⁴, R¹⁵ and R¹⁶ are independently hydrogen or a C₁-C₁₈ hydrocarbon residue containing one or more atoms/groups selected from O, N and C(O), and the sum of all carbon atoms (R¹⁴+R¹⁵+R¹⁶) is not greater than 18; or
X is CR¹⁷=CR¹⁸R¹⁹, wherein R¹⁷, R¹⁸ and R¹⁹ are independently hydrogen or a C₁-C₁₈ hydrocarbon residue, the geometry of the enol double bond is E or Z, and the sum of all carbon atoms (R¹⁷+R¹⁸+R¹⁹) is not greater than 18; or
X is —CR¹⁷═CR¹⁸R¹⁹, wherein R¹⁷, R¹⁸ and R¹⁹ are independently hydrogen or a C₁-C₁₈ hydrocarbon residue containing one or more atoms/groups selected from O, N and C(O), the geometry of the enol double bond is E or Z, and the sum of all carbon atoms (R¹⁷+R¹⁸+R¹⁹) is not greater than 18;
R¹² and R¹³ are independently hydrogen, C₁-C₆ alkyl, C₁-C₆ alkoxy residue,
—NO₂, —NH₂, —NHCO₂CH₃, —N(C₁-C₆ alkyl)₂, —N(hydroxyalkyl)₂, —NHC(O)—(C₁-C₈ alkyl) or —NHC(O)—(C₃-C₈ aryl); or
R¹² and R¹³ are attached at the positions C(vi,vii), C(vii,viii), or C(viii,ix), and form together with the carbon atoms to which they are attached a dioxolane ring or a dioxane ring;
R¹¹ in ii- or iii-position is hydrogen, C₁-C₄ alkyl, C₂-C₄ alkenyl, C₃-C₆ cycloalkyl, or —CN.

2. (canceled)

3. (canceled)

4. (canceled)

5. (canceled)

6. A process according to claim 1 for preparing compositions which provide upon activation an olfactory compound comprising incorporating into the composition a compound of formula (I) wherein R⁵, R⁶ and R⁷ are independently hydrogen or a C₁-C₁₀ hydrocarbon residue.

7. A process according to claim 1 wherein the sum of carbon atoms R⁵+R⁶+R⁷ is between 6 and 15.

8. A process according to claim 1 for preparing compositions which provide upon activation an olfactory compound comprising incorporating into the composition a compound of formula (I) wherein at least one residue R⁵, R⁶ and R⁷ is not hydrogen.

9. A process according to claim 1 for preparing compositions which provide upon activation an olfactory compound comprising incorporating into the composition a compound of formula (I) wherein R⁸, R⁹ and R¹⁰ are independently hydrogen or a C₁-C₁₀ hydrocarbon residue.

10. A process according to claim 1 wherein the sum of carbon atoms R⁵+R⁹+R¹⁰ is between 6 and 15.

11. A process according to claim 1 for preparing compositions which provide upon activation an olfactory compound comprising incorporating into the composition a compound of formula (I) wherein at least one residue R⁸, R⁹ and R¹⁰ is not hydrogen.

12. A process according to claim 1 for preparing compositions which provide upon activation an olfactory compound comprising incorporating into the composition a compound of formula (I) wherein R¹⁴, R¹⁵ and R¹⁶ are independently hydrogen or a C₁-C₁₀ hydrocarbon residue.

13. A process according to claim 1 wherein the sum of carbon atoms R¹⁴+R¹⁵+R¹⁶ is between 6 and 15.

14. A process according to claim 1 for preparing compositions which provide upon activation an olfactory compound comprising incorporating into the composition a compound of formula (I) wherein at least one residue R¹⁴, R¹⁵ and R¹⁶ is not hydrogen.

15. A process according to claim 1 for preparing compositions which provide upon activation an olfactory compound comprising incorporating into the composition a compound of formula (I) wherein R¹⁷, R¹⁸ and R¹⁹ are independently hydrogen or a C₁-C₁₀ hydrocarbon residue.

16. A process according to claim 1 wherein the sum of carbon atoms R¹⁷+R¹⁸+R¹⁹ is between 6 and 15.

17. A process according to claim 1 for preparing compositions which provide upon activation an olfactory compound comprising incorporating into the composition a compound of formula (I) wherein at least one residue R¹⁷, R¹⁸ and R¹⁹ is not hydrogen.

18. A process according to claim 1 for preparing compositions which provide upon activation an olfactory compound comprising incorporating into the composition a compound of formula (I) wherein n is 1.

19. A process according to claim 1 for preparing compositions which provide upon activation an olfactory compound comprising incorporating into the composition a compound of formula (I) wherein n is 1 and Y a residue selected from the group consisting of a fragrant alcohol HO—CR⁵R⁶R⁷, of the enol form of a fragrant aldehyde of the formula O═(CH)—CHR⁹R¹⁰, and of the enol form of a fragrant ketone of the formula O═(CR⁸)—CHR⁹R¹⁰.

20. A process according to claim 19 wherein the fragrant alcohol having a molecular weight between 46 and 400.

21. A process according to claim 1 for preparing compositions which provide upon activation an olfactory compound comprising incorporating into the composition a compound of formula (I) wherein n is 1 and Y a residue selected from the group consisting of a fragrant alcohol HO—CR⁵R⁶R⁷, of the enol form of a fragrant aldehyde of the formula O═(CH)—CHR⁹R¹⁰, and of the enol form of a fragrant ketone of the formula O═(CR⁸)—CHR⁹R¹⁰, and R is selected from the group consisting of methyl, ethyl, propyl, butyl, pentyl, 2-ethylhexyl, cyclopentyl, cyclohexyl, and the residue of a fragrant alcohol.

22. A process according to claim 21 wherein the fragrant alcohol having a molecular weight between 46 and 400.

23. A process according to claim 19 wherein the fragrant aldehyde having a molecular weight between 100 and 450.

24. A process according to claim 21 wherein the fragrant aldehyde having a molecular weight between 100 and 450.

25. A process according to claim 19 wherein the fragrant ketone having a molecular weight between 100 and 450.

26. A process according to claim 21 wherein the fragrant ketone having a molecular weight between 100 and 450.

27. A process according to claim 1 for preparing compositions which provide upon activation an olfactory compound comprising incorporating into the composition a compound of formula (I) wherein n is 1 and R is amonovalent residue of formula (i).

28. A consumer product comprising a compound of formula (I) as defined by claim 1.

29. A consumer product according to claim 28 comprising a compound of formula (I) wherein n is 1 and Y a residue selected from the group consisting of a fragrant alcohol HO—CR⁵R⁶R⁷, of the enol form of a fragrant aldehyde of the formula O═(CH)—CHR⁹R¹⁰, and of the enol form of a fragrant ketone of the formula O═(CR⁸)-CHR⁹R¹⁰.

30. A consumer product according to claim 28 wherein the consumer product is selected from fine fragrance, industrial care products, institutional care products, home care products, personal care products, industrial cleaning products, institutional cleaning products, home cleaning products, and fabric conditioner.

31. A process of providing an olfactory compound to a substrate comprising the steps:
a) cleaving a compound of formula (I) by hydrolysis 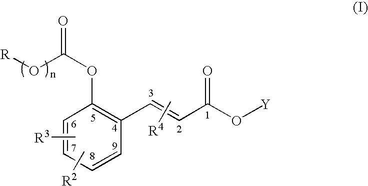
wherein the acrylic acid ester double bound is of the E configuration;
n is zero or 1;
Y is —CR⁵R⁶R⁷, wherein R⁵, R⁶ and R⁷ are independently hydrogen or a C₁-C₁₈ hydrocarbon residue, and the sum of all carbon atoms (R⁵+R⁶+R⁷) is not greater than 18; or
Y is —CR⁵R⁶R⁷, wherein R⁵, R⁶ and R⁷ are independently hydrogen or a C₁-C₁₈ hydrocarbon residue containing one or more atoms/groups selected from O, N and C(O), and the sum of all carbon atoms (R⁵+R⁶+R⁷) is not greater than 18; or
Y is —CR⁸═CR⁹R¹⁰, wherein R⁸, R⁹ and R¹⁰ are independently hydrogen or a C₁-C₁₈ hydrocarbon residue, the geometry of the enol double bond is E or Z, and the sum of all carbon atoms (R⁸+R⁹+R¹⁰) is not greater than 18; or
Y is —R⁸═CR⁹R¹⁰, wherein 18, 19 and R¹⁰ are independently hydrogen or a C₁-C₁₈ hydrocarbon residue containing one or more atoms/groups selected from O, N and C(O), the geometry of the enol double bond is E or Z, and the sum of all carbon atoms (R⁸+R⁹+R¹⁰) is not greater than 18;
R² and R³ are independently hydrogen, C₁-C₆ alkyl, C₁-C₆ alkoxy residue, —NH₂, —NO₂, —NHCO₂CH₃, —N(C₁-C₆ alkyl)₂, —N(hydroxyalkyl)₂, —NHC(O)-(C₁-C₈ alkyl) or —NHC(O)—(C₃-C₈ aryl); or
R² and 13 are attached at the positions C(6,7), C(7,8), or C(8,9), and form together with the carbon atoms to which they are attached a dioxolane ring or a dioxane ring;
R⁴ in 2- or 3-position is hydrogen, C₁-C₄ alkyl, C₂-C₄ alkenyl, C₃-C₆ cycloalkyl, or CN; and
a) if n is zero, R is a C₁-C₂₄ hydrocarbon residue, or C₁-C₂₋₄ hydrocarbon residue containing one or more heteroatoms selected from N, O and Si; or
b) if n is 1, R is a C₁-C₂₅ hydrocarbon residue, a C₁-C₂₅ hydrocarbon residue containing one or more atoms/groups selected from N, O, Si, and C(O), or C₁-C₂₅ hydrocarbon residue substituted by an ionic substituent of the formula N(R²⁰)₃⁺, in which R²⁰ is the residue of an alkyl group with 1 to 18 carbon atoms; or
R is a monovalent residue of the formula (I) 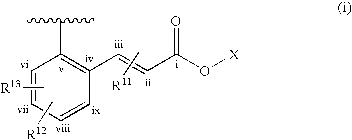
wherein
X is —CR¹⁴R¹⁵R¹⁶, wherein R¹⁴, R¹⁵ and R¹⁶ are independently hydrogen or a C₁-C₁₈ hydrocarbon residue, and the sum of all carbon atoms (R¹⁴+R¹⁵+R¹⁶) is not greater than 18; or
X is —CR¹⁴R¹⁵R¹⁶, wherein R⁴, R¹⁵ and R⁶ are independently hydrogen or a C₁-C₁₈ hydrocarbon residue containing one or more atoms/groups selected from O, N and C(O), and the sum of all carbon atoms (R¹⁴+R¹⁵+R¹⁶) is not greater than 18; or
X is CR⁷═CR¹⁸R¹⁹, wherein R¹⁷, R¹⁸ and R¹⁹ are independently hydrogen or a C₁-C₁₈ hydrocarbon residue, the geometry of the enol double bond is E or Z, and the sum of all carbon atoms (R¹⁷+R¹⁸+R¹⁹) is not greater than 18; or
X is —CR¹⁷=CR⁸R⁹, wherein R¹⁷, R¹⁸ and R¹⁹ are independently hydrogen or a C₁-C₁₈ hydrocarbon residue containing one or more atoms/groups selected from O, N and C(O), the geometry of the enol double bond is E or Z, and the sum of all carbon atoms (R¹⁷+R¹⁸+R¹⁹) is not greater than 18;
R¹² and R¹³ are independently hydrogen, C₁-C₆ alkyl, C₁-C₆ alkoxy residue,
—NO₂, —NH₂, —NHCO₂CH₃, —N(C₁-C₆ alkyl)₂, —N(hydroxyalkyl)₂,
—NHC(O)—(C₁-C₈ alkyl) or —NHC(O)—(C₃-C₈ aryl); or
R¹² and R¹³ are attached at the positions C(vi,vii), C(vii,viii), or C(viii,ix), and form together with the carbon atoms to which they are attached a dioxolane ring or a dioxane ring;
R¹¹ in ii- or iii-position is hydrogen, C₁-C₄ alkyl, C₂-C₄ alkenyl, C₃-C₆ cycloalkyl, or —CN;
resulting in a compound of formula (Ia) 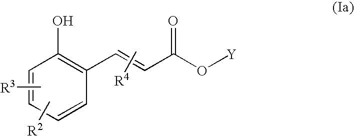
wherein R², R³, R⁴ and Y have the same meaning as given above; followed by
b) cleaving the compound of formula (Ia) of step a under activating conditions in the presence of light resulting in a coumarin (IIa) 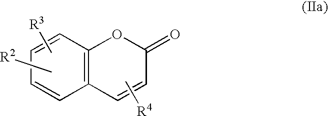
wherein R², R³ and R⁴ have the same meaning as given above.

32. A compound of formula (I) 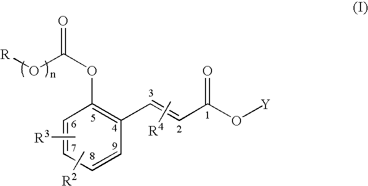
wherein the acrylic acid ester double bound is of the E configuration;
n is zero or 1;
Y is CR⁵R⁶R⁷, wherein R⁵, R⁶ and R⁷ are independently hydrogen or a C₁-C₁₈ hydrocarbon residue, and the sum of all carbon atoms (R⁵+R⁶+R⁷) is not greater than 18 and at least 6; or
Y is —CR⁵R⁶R⁷, wherein R⁵, R⁶ and R⁷ are independently hydrogen or a C₁-C₁₁ aliphatic residue containing one or more atoms/groups selected from O, N and C(O), the sum of all carbon atoms (R⁵+R⁶+R⁷) is not greater than 18; and at least one of the residues R⁵, R⁶ and R⁷ is not hydrogen; or
Y is —CR⁸═CR⁹R¹⁰, wherein R⁸, R⁹ and R¹⁰ are independently hydrogen or a C₁-C₁₈ hydrocarbon residue, the geometry of the enol double bond is E or Z, and the sum of all carbon atoms (R⁸+R⁹+R¹⁰) is not greater than 18; or
Y is —CR⁸═CR⁹R¹⁰, wherein R⁸, R⁹ and R¹⁰ are independently hydrogen or a C₁-C₁₈ hydrocarbon residue containing one or more atoms/groups selected from O, N and C(O), the geometry of the enol double bond is E or Z, and the sum of all carbon atoms (R⁸+R⁹+R¹⁰) is not greater than 18;
R² and R³ are independently hydrogen, C₁-C₆ alkyl, C₁-C₆ alkoxy residue, —NH₂, —NO₂₁—NHCO₂CH₃, —N(C₁-C₆ alkyl)₂, —N(hydroxyalkyl)₂, —NHC(O)—(C₁-C₈ alkyl) or
—NHC(O)—(C₃-C₈ aryl); or
R² and R³ are attached at the positions C(6,7), C(7,8), or C(8,9), and form together with the carbon atoms to which they are attached a dioxolane ring or a dioxane ring;
R⁴ in 2- or 3-position is hydrogen, C₁-C₄ alkyl, C₂-C₄ alkenyl, C₃-C₆ cycloalkyl, or —CN; and
a) if n is zero, R is a C₂-C₂₄ hydrocarbon residue, or C₁-C₂₄ hydrocarbon residue containing one or more heteroatoms selected from N, O and Si; or
b) if n is 1, R is a C₁-C₂₅ hydrocarbon residue, a C₁-C₂₅ hydrocarbon residue containing one or more atoms/groups selected from N, O, Si, and C(O), or C₁-C₂₅ hydrocarbon residue substituted by an ionic substituent of the formula N(R²⁰)₃⁺, in which R²⁰ is the residue of an alkyl group with 1 to 18 carbon atoms; or
R is a monovalent residue of the formula (I) 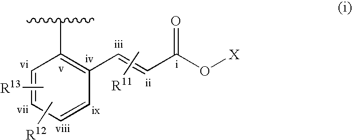
wherein
X is —CR¹⁴R¹⁵R¹⁶, wherein R¹⁴, R¹⁵ and R¹⁶ are independently hydrogen or a C₁-C₁₈ hydrocarbon residue, and the sum of all carbon atoms (R¹⁴+R¹⁵+R¹⁶) is not greater than 18; or
X is —CR¹⁴R¹⁵R¹⁶, wherein R¹⁴, R¹⁵ and R¹⁶ are independently hydrogen or a C₁-C₁₈ hydrocarbon residue containing one or more atoms/groups selected from O, N and C(O), and the sum of all carbon atoms (R¹⁴+R¹⁵+R¹⁶) is not greater than 18; or
X is —CR¹⁷═CR¹⁸R¹⁹, wherein R¹⁷, R¹⁸ and R¹⁹ are independently hydrogen or a C₁-C₁₈ hydrocarbon residue, the geometry of the enol double bond is E or Z, and the sum of all carbon atoms (R¹⁷+R¹⁸+R¹⁹) is not greater than 18; or
X is —CR¹⁷=CR¹⁸R¹⁹, wherein R¹⁷, R¹⁸ and R¹⁹ are independently hydrogen or a C₁-C₁₈ hydrocarbon residue containing one or more atoms/groups selected from O, N and C(O), the geometry of the enol double bond is E or Z, and the sum of all carbon atoms (R¹⁷+R¹⁸+R¹⁹) is not greater than 18;
R¹² and R¹³ are independently hydrogen, C₁-C₆ alkyl, C₁-C₆ alkoxy residue,
—NO₂, —NH₂, —NHCO₂CH₃, —N(C₁-C₆ alkyl)₂, —N(hydroxyalkyl)₂,
—NHC(O)—(C₁-C₉ alkyl) or —NHC(O)—(C₃-C₈ aryl); or
R¹² and R¹³ are attached at the positions C(vi,vii), C(vii,viii), or C(viii,ix), and form together with the carbon atoms to which they are attached a dioxolane ring or a dioxane ring;
R¹¹ in ii- or iii-position is hydrogen, C₁-C₄ alkyl, C₂-C₄ alkenyl, C₃-C₆ cycloalkyl, or —CN.

33. A compound according to claim 32 wherein n is 1 and R², R³ and R⁴ are hydrogen.

34. A compound according to claim 32 wherein n is 1, R² and R³ are hydrogen and R⁴ is selected from methyl and —CN.

35. A compound according to claim 32 wherein n is 1, R² and R³ are hydrogen and R⁴ is phenyl at C(3).

36. A compound according to claim 32 wherein n is 1, 2 is hydrogen, R³ is at either C(6) to C(8) and R³ is selected from methyl, ethyl, propyl, isopropyl, methoxy, ethoxy and propyloxy, and R⁴ is selected from hydrogen, methyl, —CN, and phenyl at C(3).

37. A compound according to claim 32 wherein n is 1, R² and R³ are methyl at position C(6,7), C(6,8), C(6,9), C(7,8), or C(9,9), and R⁴ is selected from hydrogen, —CN, and phenyl at C(3).

38. A compound according to claim 32 wherein n is 1, R² and R³ are methoxy at C(7,9), and R⁴ is selected from hydrogen, —CN, and phenyl at C(3).

39. A compound according to claim 32 wherein n is 1, R⁴ is selected from hydrogen, methyl, —CN, and phenyl at C(3), R² is methyl at C(6) and R³ is isopropyl at C(9).

40. A compound according to claim 32 wherein n is 1, R⁴ is selected from hydrogen, methyl, —CN, and phenyl at C(3), R² is isopropyl at C(6) and R³ is methyl at C(9).

41. A compound according to claim 32 wherein n is 1, R⁴ is selected from hydrogen and methyl, and R² and R³ are hydrogen.

42. A compound according to claim 32 wherein n is 1, R⁴ is selected from hydrogen and methyl, R² is hydrogen, and R³ is 7-methoxy.

43. A compound according to claim 32 wherein n is 1, R⁴ is selected from hydrogen and methyl, R² is hydrogen, and R³ is 6-methyl.

44. A compound according to claim 32 wherein n is 1, R⁴ is selected from hydrogen and methyl, R² is hydrogen, and R³ is 7-methyl.

45. A compound according to claim 32 wherein n is 1, R⁴ is selected from hydrogen and methyl, R² is hydrogen, and R³ is 8-methyl.

46. A compound according to claim 32 wherein n is 1, R⁴ is selected from hydrogen and methyl, R² is hydrogen, and R³ is 6-tert-butyl.

47. A compound according to claim 32 wherein n is 1, R⁴ is selected from hydrogen and methyl, R² is 6-tert-butyl, and R³ is 8-tert-butyl.

48. A compound according to claim 32 wherein n is 1 and R is a monovalent residue of the formula (I) 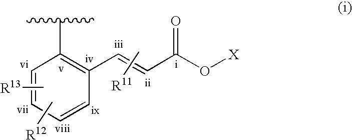
wherein X, R¹¹, R¹² and R¹³ are defined according to claim 27.

49. A compound according to claim 48 wherein R⁴═R¹¹, R³═R¹³ and R²═R¹².

50. A process according to claim 31 wherein the compound of formula (I) hydrolysis in the presence of enzymes.
